(19) United States
(12) Patent Application Publication
(10) Pub. No.: US 2026/0340578 A1
(43) Pub. Date: Oct. 29, 2026

(54) OPHTHALMIC SURGICAL SUPPLY MANAGEMENT SYSTEM

(71) Applicant: Alcon Inc., Fribourg (CH)

(72) Inventors: Vidhya Mellarkod, Irving, TX (US); Raja Chhabeda, Aledo, TX (US)

(21) Appl. No.: 19/577,502

(22) Filed: Mar. 25, 2026

Related U.S. Application Data
(60) Provisional application No. 63/794,493, filed on Apr. 25, 2025.

(51) Int. Cl. G06Q 10/087; G06K 7/10

(52) U.S. Cl. CPC G06Q 10/08726; G06K 7/10366

(57) ABSTRACT

The present disclosure relates to operations that include obtaining surgery data about surgeries scheduled for a surgical center. The operations may further include identifying, based on an analysis of the surgery data, potential surgical supplies to be used during the surgeries. The operations may further include performing a radio-frequency identification (RFID) scan that scans individual RFID tags corresponding to individual surgical supply items to obtain inventory data about an inventory of surgical supplies at the surgical center. The operations may further include identifying depleted surgical supplies for the surgeries based on the inventory data and the potential surgical supplies. The operations may further include performing one or more inventory management actions based on the identified depleted surgical supplies.

### CROSS-REFERENCE TO RELATED APPLICATIONS

This application claims priority to and benefit of U.S. Provisional Patent Application No. 63/794,493, filed Apr. 25, 2025, which is incorporated by reference herein in its entirety, and is hereby expressly made a part of this specification.

### FIELD

The present disclosure relates to an ophthalmic surgical supply management system.

### BACKGROUND

Surgical centers may be specialized healthcare facilities where outpatient surgeries and related medical services are provided. Surgical centers may operate independently or in affiliation with one or more hospitals. A wide variety of surgical procedures may be offered at a surgical center. For example, ophthalmic surgical centers may offer cataract surgeries, glaucoma surgeries, retinal surgeries, and corneal surgeries, among others.

Accordingly, surgical centers may stock hundreds, thousands, or even tens of thousands of different types of surgical supplies. The inventory of such surgical supplies may change frequently as various surgeries are conducted. Surgical centers may face challenges in maintaining appropriate inventory levels of surgical supplies while reducing overstocking and waste.

Further, tens, hundreds, or even thousands of different surgeries may be scheduled to be performed at a surgical center each month. Scheduling surgeries at surgical centers may be a complex task that may require consideration of multiple scheduling factors such as patient needs, surgeon availability, equipment requirements, and facility constraints. Traditional manual surgical scheduling management methods may be time-consuming and error-prone and may result in reduced utilization of surgical center resources.

The subject matter claimed herein is not limited to embodiments that solve any disadvantages or that operate only in environments such as those described above. Rather, this background is only provided to illustrate one example technology area where some embodiments described herein may be practiced.

### SUMMARY

The present disclosure relates to operations that include obtaining surgery data about surgeries scheduled for a surgical center. The operations may further include identifying potential surgical supplies to be used during the surgeries based on an analysis of the surgery data. The operations may also include obtaining inventory data about an inventory of surgical supplies at the surgical center by performing a radio-frequency identification (RFID) scan that scans individual RFID tags corresponding to individual surgical supply items. The operations may include identifying depleted surgical supplies for the surgeries based on the inventory data and the potential surgical supplies. The operations may further include generating one or more inventory management actions based on the identified depleted surgical supplies.

The objects and advantages of the embodiments will be realized and achieved at least by the elements, features, and combinations particularly pointed out in the claims. It is to be understood that both the foregoing general description and the following detailed description are explanatory and are not restrictive of the invention, as claimed.

### BRIEF DESCRIPTION OF THE DRAWINGS

The present disclosure relates to methods and systems for ophthalmic surgical supply inventory and schedule management, wherein:

FIG. 1 is a block diagram of an example inventory management system for managing surgical supply inventory, according to one or more embodiments of the present disclosure;

FIG. 2 illustrates an example process that may be performed to generate inventory management actions, according to one or more embodiments of the present disclosure;

FIG. 3 illustrates an example inventory identification system, according to one or more embodiments of the present disclosure;

FIG. 4 is a flow diagram illustrating a method for surgical supply inventory management, according to one or more embodiments of the present disclosure;

FIG. 5 is a block diagram of an example surgery scheduling system that may be used with and/or implement one or more embodiments of the present disclosure related to surgical schedule management;

FIG. 6 illustrates an example process that may be performed to generate a surgical schedule, according to one or more embodiments of the present disclosure;

FIG. 7A is a block diagram of generating a surgical schedule using a room scheduling operation on surgery data, surgical center data, and priority scores, according to one or more embodiments of the present disclosure;

FIG. 7B is a flow diagram illustrating a method of room scheduling, according to one or more embodiments of the present disclosure;

FIG. 8 is a flow diagram illustrating a method for surgical schedule management, according to one or more embodiments of the present disclosure;

FIG. 9A illustrates an example surgical supply inventory and schedule management system, according to one or more embodiments of the present disclosure;

FIGS. 9B-9D illustrate example graphical user interfaces (GUIs) of a surgical supply inventory and schedule management system, according to one or more embodiments of the present disclosure; and

FIG. 10 is a block diagram of an example computing system suitable for use in implementing one or more embodiments of the present disclosure.

### DETAILED DESCRIPTION

Surgical centers may face numerous challenges with respect to coordinating surgeries and/or other related services. For example, surgical centers may struggle with maintaining accurate, real-time accounts of surgical supply inventory. Traditional inventory management methods may rely on manual processes for tracking, verifying, and/or ordering supplies. Such manual processes may be time-consuming and prone to errors. For example, surgical center staff members may manually compile lists of surgical supplies for upcoming surgeries, which may be labor-intensive. Verification of surgical supplies for each surgery may involve multiple manual checks, potentially increasing stress and the risk of mistakes. Ordering surgical supplies to restock depleted surgical supplies may be inefficient and result in overstocking and/or stockouts of surgical supplies when based on inaccurate inventory.

One or more embodiments of the present disclosure may generally relate to a surgical supply inventory management system that provides an automated approach to managing the inventory of surgical supplies, such as ophthalmic surgical supplies. In some embodiments, the surgical supply inventory management system may integrate data from multiple sources to generate lists of surgical supplies, compare anticipated supplies to current inventory, assist with ordering surgical supplies, verify surgical supplies for specific surgeries, update inventory tracking in real-time, enable just-in-time ordering, etc. In some embodiments, the system may include a combination of hardware and software elements. For example, the system may include one or more computing systems that interface with a wireless identification system, such as a radio-frequency identification (RFID) system, for inventory tracking and/or predicting surgical supply use (e.g., techniques using artificial intelligence (AI)), which may improve inventory management. For example, in some embodiments, the system may improve efficiency, reduce errors, and/or provide a better accounting of surgical supply inventory across a surgical center, such as an ophthalmic surgical center.

For example, ophthalmic surgical centers may offer a variety of surgeries such as refractive surgeries for vision correction (e.g., Laser-Assisted in Situ Keratomileusis (LASIK)), cataract surgeries (e.g., phacoemulsification with intraocular lens (IOL) implantation) and femtosecond laser-assisted cataract surgery (FLACS)), glaucoma surgeries (e.g., minimally invasive glaucoma surgery (MIGS)), etc.). As such, a surgical supply inventory management system according to one or more embodiments of the present disclosure may be implemented with respect to ophthalmic surgical centers to assist staff members, nurses, and/or surgeons with ensuring proper ophthalmic surgical supplies are available for various surgeries (e.g., ophthalmic surgical supplies may include IOLs, viscoelastic agents, silicone oil, silicone gas, vitrectomy cutters, phacoemulsification handpieces and tubing, etc.). The wide variety of specialized surgical supplies used in ophthalmic surgeries may make surgical supply inventory management particularly complex in ophthalmic surgical center contexts. Additionally, ophthalmic surgical centers may face challenges in predicting surgical supplies based on surgeon preferences, procedure-specific requirements, and/or facility protocols.

In some instances, ophthalmic surgical centers are presented with unique challenges in surgical supply inventory management compared to other types of surgical centers because ophthalmic procedures, such as cataract surgeries, are generally short in length (e.g., 10 to 30 minutes). As a result, ophthalmic surgical centers may handle a relatively high volume of surgeries any given day and may correspondingly consume a large number of surgical supplies. Furthermore, the specialized nature of eye-related surgeries may further increase the difficulty of surgical supply inventory management for ophthalmic surgical centers. For example, ophthalmic surgical supplies may be temperature-dependent (e.g., viscoelastic agents used in cataract surgeries may generally be stored at between 2 °C and 8 °C) and/or may require specific storage conditions.

Additionally, ophthalmic surgical centers that perform surgeries involving the implantation of one or more IOLs may be unique from other types of surgical centers with typical surgical supply inventory concerns because IOLs are generally provided on consignment (e.g., an ophthalmic surgical center stores a supply of IOLs provided by a manufacturer/distributor without paying for the IOLs upfront but rather pays for the lenses once the lenses have been used in a surgery). Managing surgical supply inventory on consignment may be useful to ophthalmic surgical centers for surgical supplies such as IOLs because such surgical supplies generally come in a wide range of prescriptions, are high-cost items (e.g., around $1,000-$4,000 each), and may be patient-specific. However, managing surgical supply inventory on consignment may also involve additional administrative burden to track and/or verify which supplies have been used (e.g., to determine which supplies may be expired and/or close to expiring, to determine which supplies may need to be restocked, to determine which supplies need to be paid for, etc.).

Therefore, one or more embodiments of the present disclosure may relate to surgical supply inventory management related to ophthalmic surgical centers. In particular, in some embodiments, surgical supplies in an ophthalmic surgical center may be identified from a RFID scan of surgical supply packaging corresponding to individual surgical supplies with RFID tags within the ophthalmic surgical center and/or within a particular inventory room or operating room of the ophthalmic surgical center.

In these and other embodiments, one or more inventory management actions may be performed with respect to the identified surgical supplies. In some embodiments, the inventory management actions that may be performed with respect to the identified surgical supplies may include a variety of operations to enhance efficiency and/or accuracy within the ophthalmic surgical center. For example, the inventory management actions may include generating inventory reports (e.g., real-time inventory reports) that may provide a comprehensive view of available surgical supplies, including quantities, locations, expiration dates, and/or any other information about the surgical supplies. In some embodiments, the inventory management actions may include generating inventory reports that may be customized based on user preferences. In these and other embodiments, inventory reports may be accessible through a graphical user interface (GUI) of the surgical supply inventory management system. In some embodiments, the inventory management actions may also include automatically updating inventory status designations for surgical supplies, such as transitioning items from “available” to “allocated” when assigned to a particular surgery, and further transitioning to “used” once the surgical supplies have been used in a surgery.

In some embodiments, the inventory management actions may include generating alerts for surgical supplies that are approaching their expiration dates. In some embodiments, these alerts may be configured with customizable thresholds (e.g., alerts indicating which surgical supplies expire within the next 30 days, 60 days, 180 days, etc.) to provide advance notice for taking appropriate actions (e.g., actions such as prioritizing the use of soon-to-expire surgical supplies).

In some embodiments, the system may generate and/or perform inventory management actions related to consignment inventory tracking (e.g., recording usage of consigned surgical supplies, generating reports for billing purposes, facilitating communication with suppliers regarding consignment inventory levels, etc.).

In some embodiments, the inventory management actions may include automated reordering processes triggered by predefined inventory thresholds and/or by the use of surgical supplies during surgeries. For example, the inventory management actions may include generating purchase orders that may be automatically sent to suppliers or queued for approval by an authorized staff member at the surgical center. In some embodiments, the inventory management actions may include one or more order tracking actions. For example, the inventory management actions may include monitoring the status of placed orders and/or estimating delivery dates.

In some embodiments, the inventory management actions may include facilitating the physical retrieval of surgical supplies. For example, the inventory management actions may include activation of location indicators, such as lights that may be illuminated to guide surgical center staff members to the correct storage locations. In some embodiments, location indicators may be color-coded to correspond to specific surgeries and/or users, which may streamline the surgical supply retrieval process.

In some embodiments, the inventory management actions may include verifying that specific surgical supplies have been pulled for specific surgeries. For example, one or more additional RFID scans may be performed to verify which surgical supplies were pulled.

In some embodiments, the inventory management actions may include generating predictive analytics reports (e.g., based on historical use patterns, scheduled surgeries, surgeon preferences, etc.). In some embodiments, predictive analytics reports may provide insights into anticipated surgical supply needs, potential inventory shortages, and/or recommended ordering strategies (e.g., ordering strategies that may reduce costs). In some embodiments, the inventory management actions may include reconciliation processes that may compare actual usage of surgical supplies with predicted usage to identify discrepancies and that may refine future predictions.

In some embodiments, the inventory management actions may include facilitating returns of unused surgical supplies to inventory, which may involve updating inventory records and/or adjusting status designations. In some embodiments, the inventory management actions may include one or more actions related to returning surgical supplies to suppliers (e.g., contingent inventory, surgical supplies received in error, etc.). In some embodiments, the inventory management actions may include quality control actions, such as recording and/or tracking any issues identified with surgical supplies. In these and other embodiments, the inventory management actions may include one or more actions for facilitating communication with suppliers regarding issues with surgical supplies.

The embodiments of the present disclosure may be used with any suitable system, apparatus, or device in which such surgical supply inventory management may be beneficial. For example, in some embodiments, a surgical supply inventory management system may be configured to perform inventory identification and/or inventory assessment and may be configured to also generate inventory management actions with respect to surgical supplies in an ophthalmic surgical center. Such inventory management actions may allow for the ophthalmic surgical center to more accurately manage current surgical supply inventory, which may accordingly result in an improvement in the accuracy and/or efficiency of ophthalmic surgical center operation. In some embodiments of the present disclosure, surgical supply inventory management may accordingly improve the efficacy and/or safety of ophthalmic surgical centers. In some embodiments, addressing surgical supply inventory management challenges may also be beneficial in reducing costs and/or in enhancing patient care in ophthalmic surgical centers.

One or more embodiments of the present disclosure may also relate to systems and methods for automated scheduling of surgical procedures. The scheduling of surgeries in surgical centers may present numerous challenges. For example, scheduling personnel may need to consider various factors when creating a patient flow schedule for each surgery day. These factors may include patient health considerations, case complexity, surgeon preferences, clinic preferences, patient preferences, surgery center preferences, operating room limitations, and equipment availability.

Manually creating a surgical schedule while accounting for all of these factors may be a time-consuming and complex process. Scheduling personnel may require several hours to generate a surgical schedule, which may continue to change. This may result in surgery centers waiting until the last day to finalize schedules and inform patients, often not until the evening before the day a surgery is to be performed.

The manual scheduling process may be prone to human error and inefficiencies. A scheduler may need to review and balance multiple criteria simultaneously, which may be challenging manually. Manual scheduling may have limited ability to account for historical data on actual procedure durations to improve future scheduling accuracy.

Furthermore, manual scheduling processes may provide limited ability to balance competing priorities, such as increasing operating room utilization while also accommodating surgeon preferences and/or patient needs. Traditional surgical schedule management may also face challenges in coordinating schedules across multiple operating rooms and/or procedure areas in generating a surgical schedule that may be useful for a surgical center as a whole.

Additionally, existing scheduling software solutions may allow posting of schedules once created but may not provide capabilities for automatically generating surgical schedules based on multiple complex criteria. The lack of automated scheduling tools that may handle numerous interrelated factors may lead to reduced surgical center resource utilization and/or scheduling inefficiencies.

The nature of manual scheduling may make it difficult to rapidly adjust surgical schedules in response to last-minute changes and/or cancellations. Manual rescheduling to fill gaps may be time-consuming and may result in poor resource allocation. Manual scheduling processes may have limited ability to analyze historical scheduling and/or surgery data to identify opportunities to improve surgical schedule management at a surgical center.

Further, lack of integration between surgical scheduling and surgical equipment data may create inefficiencies in equipment utilization. Manual scheduling processes may not effectively account for equipment maintenance schedules, calibration requirements, and/or recommended usage patterns to increase surgical equipment longevity.

Systems and methods of the present disclosure may provide for an automated scheduling system that may help address at least some of the challenges of manual scheduling through weighted criteria, learning from historical data, and/or optimizing across multiple constraints. By leveraging weighted criteria, historical data, and/or optimization across multiple constraints, the automated scheduling system may generate more efficient surgical schedules compared to manual scheduling methods. The learning capabilities may allow the system to continuously improve scheduling accuracy over time. This approach may help improve utilization of surgical center resources while accounting for the complex set of scheduling factors that may be involved in surgical scheduling.

Further, an automated scheduling system may be used to improve the use and/or longevity of surgical equipment itself in a manner that may be difficult or impossible for manual scheduling to accomplish. For example, the automated scheduling system may help improve equipment longevity by incorporating load balancing using surgical equipment usage information (e.g., frequency of use and/or duration of use) and surgical equipment maintenance requirements. For instance, in the context of ophthalmic surgery, the scheduling system may track the usage hours and maintenance schedules for different pieces of surgical equipment, such as phacoemulsification handpieces, femtosecond lasers, and other specialized devices. When assigning surgeries to operating rooms, the system may consider the cumulative usage hours of each piece of surgical equipment and attempt to distribute scheduling surgeries evenly across multiple similar pieces of surgical equipment (e.g., pieces of surgical equipment all of the same type of surgical equipment).

For example, in an instance in which there are three phacoemulsification handpieces available, the automated surgical scheduling system may alternate between the three phacoemulsification handpieces when scheduling cataract surgeries, rather than scheduling the same handpiece repeatedly. Such load balancing may help prevent excessive wear on any single piece of surgical equipment.

Additionally or alternatively, the automated surgical scheduling system may incorporate manufacturer-recommended usage limits and/or maintenance intervals for each piece of surgical equipment. As a piece of surgical equipment approaches a recommended service interval or usage limit, the automated surgical scheduling system may begin to preferentially assign that piece of surgical equipment for use in shorter and/or less complex surgeries. Such preferential assignment may help pieces of surgical equipment avoid being used beyond recommended amounts between receiving maintenance.

In these and other embodiments, the automated surgical scheduling system may also learn from historical data corresponding to surgical equipment performance and/or reliability. For example, in instances in which a particular piece of surgical equipment has required more frequent repairs and/or shown signs of decreased efficiency, the automated surgical scheduling system may reduce the utilization rate of that particular piece of surgical equipment (e.g., compared to newer and/or more reliable pieces of surgical equipment), which may help extend the functional lifespan of surgical equipment by dynamically adjusting usage patterns based on real-world performance data.

By distributing surgical procedures across available surgical equipment and considering factors like usage hours, maintenance schedules, and historical data, the automated surgical scheduling system may improve surgical equipment utilization in a way that promotes longevity and reduces wear. Thus, the automated surgical scheduling system may lead to reduced surgical equipment replacement and maintenance costs for a surgical center.

The embodiments of the present disclosure will be explained with reference to the accompanying figures. It is to be understood that the figures are diagrammatic and schematic representations of such example embodiments, and are not limiting, nor are they necessarily drawn to scale. In the figures, features with like numbers indicate like structure and function unless described otherwise. Further, one or more of the figures and accompanying descriptions are given with respect to performance of distortion compensation in the context of ophthalmic surgical systems. However, such uses are not meant to be limiting such that the distortion compensation described may be used in any number of different contexts and applications where it may be helpful or applicable.

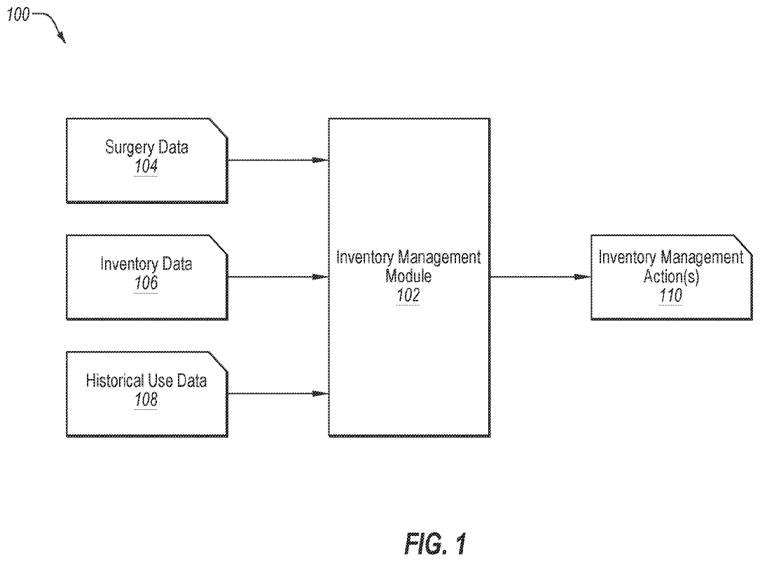

FIG. 1 is a block diagram of an example inventory management system 100 (“system 100”) that may be configured to manage surgical supply inventory (e.g., inventory of ophthalmic surgical supplies) according to one or more embodiments of the present disclosure. In general, the system 100 may be configured to perform one or more inventory management operations with respect to performance of surgical supply inventory management at a surgical center, which may include an ophthalmic surgical center in some embodiments. In some embodiments, the system 100 may include an inventory management module 102 that may be configured to generate one or more inventory management actions 110 using one or more of surgery data 104, inventory data 106, and historical use data 108.

The inventory management module 102 may include any suitable system, apparatus, or device that may be configured to manage the surgical supply inventory of one or more surgical centers. In some embodiments, the inventory management module 102 may include one or more computing systems configured to process and analyze data (e.g., surgery data 104, inventory data 106, historical use data 108, etc.) from multiple sources (e.g., from surgery scheduling systems, electronic medical record systems, financial accounting systems, etc.). In some embodiments, the inventory management module 102 may include one or more different types of devices and/or systems configured to obtain the surgery data 104, the inventory data 106, and/or the historical use data 108. For example, the inventory management module 102 may include a computing system configured to have the surgery data 104, the inventory data 106, and/or the historical use data 108 downloaded to, uploaded to, transmitted to, and/or created on the computing system.

For example, the inventory management module 102 may be implemented using hardware including one or more processors, central processing units (CPUs), graphics processing units (GPUs), data processing units (DPUs), parallel processing units (PPUs), microprocessors (e.g., to perform or control performance of one or more operations), field-programmable gate arrays (FPGAs), application-specific integrated circuits (ASICs), accelerators (e.g., deep learning accelerators (DLAs)), and/or other processor types. In these and other embodiments, the inventory management module 102 may be implemented using a combination of hardware and software.

Additionally or alternatively, the inventory management module 102 may include one or more artificial intelligence (AI) systems. In some embodiments, an AI system included in the inventory management module 102 may be configured to detect patterns in surgical supply usage and/or to predict future inventory use using one or more AI models. For example, the inventory management module 102 may use one or more neural networks, deep learning models, transformer-based models, natural language processing (NLP) models, reinforcement learning models, etc. to transform surgery data 104, inventory data 106, and/or historical use data 108 into one or more inventory management actions 110.

In some embodiments, the inventory management module 102 may be configured to obtain surgery data 104. In some embodiments, the surgery data 104 may include any suitable type of data. For example, the surgery data 104 may include numerical data, character strings that include characters such as letters, symbols, or other characters, a combination of numbers and characters, images, and/or other types of data. In some embodiments, the surgery data 104 may generally include information corresponding to surgeries at an ophthalmic surgical center. In some embodiments, the surgery data 104 may include information about the types of surgeries to be performed, such as a type of ophthalmic surgery (e.g., cataract surgery, corneal transplant, vitreoretinal surgery, etc.). In these and other embodiments, the surgery data 104 may include information about the surgical team assigned to the respective surgeries. For example, the names and roles of one or more surgeons, anesthesiologists, nurses, and/or other staff members involved in individual surgeries may be included in the surgery data 104.

Additionally or alternatively, the surgery data 104 may include information about the preferences of the surgical team members. For example, the surgery data 104 may include information such as preferred surgical techniques, equipment, and/or supplies (e.g., preferred brand of supplies) of the surgical team members.

In these and other embodiments, the surgery data 104 may include patient information. For example, the surgery data 104 may include information about a respective patient's age, medical history, allergies, etc. and which surgery is being performed for such patient by which surgical team. Additionally or alternatively, the patient information may include specific information about the patients that may be pertinent to their respective surgeries, such as the type of IOL and corresponding characteristics to be used for specific patients.

In some embodiments, the surgery data 104 may include information about the surgical center. For example, the surgery data 104 may include information about available operating rooms and/or equipment in the surgical center at a particular time. In some embodiments, the surgery data 104 may be updated in real-time to reflect surgical schedule changes that may occur in a surgical center. For example, information about additions, cancellations, and/or other modifications to scheduled surgeries may be included in the surgery data 104.

In some embodiments, the inventory management module 102 may be configured to obtain inventory data 106. In some embodiments, the inventory data 106 may include any information corresponding to one or more surgical supplies.

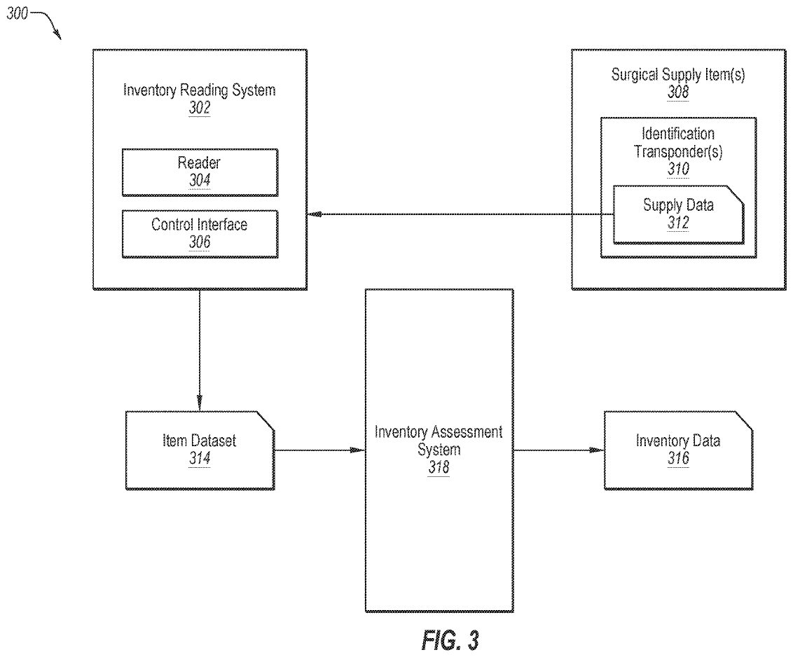

For example, in some embodiments, the inventory data 106 may include surgical supply stock level information. For instance, the inventory data 106 may include information about how many IOLs are currently stocked in an ophthalmic surgical center. Additionally or alternatively, the inventory data 106 may include surgical supply expiration date information. For example, information about how many consigned IOLs have expired and/or about how many IOLs will expire in a certain timeframe (e.g., expire in 0-30 days, expire in 31-60 days, etc.) may be included in the inventory data 106. In some embodiments, the inventory data 106 may be based on and/or include surgical supply product identifier information (e.g., unique identifier information that may be obtained via RFID tags, universal product codes (UPCs), stock keeping units (SKUs), quick response (QR) codes, health industry bar codes (HIBCs), and/or any other type of product identifier used for surgical supplies). In some embodiments, the inventory data 106 may be generated using one or more elements of an inventory identification system, such as that described below with respect to FIG. 3.

In these and other embodiments, the inventory management module 102 may be configured to obtain historical use data 108. In some embodiments, the historical use data 108 may be obtained by the inventory management module 102 via a network (e.g., the network 708 described below with respect to FIG. 12A). In some embodiments, the historical use data 108 may be generated from electronic medical records (EMRs), surgery case logs, inventory management databases, billing systems, and/or any other system that may be configured to track surgical supplies used during previous surgeries.

In some embodiments, the historical use data 108 may include any information corresponding to previously performed surgeries. Additionally or alternatively, the historical use data 108 may include information about the surgical supplies that were used during previously performed surgeries. For example, the historical use data 108 may include information about the number and type of IOLs used during previous ophthalmic surgeries.

In these and other embodiments, the historical use data 108 may include surgeon-specific surgical supply use information. For example, the historical use data 108 may include information about how many/how much of certain surgical supplies a given surgeon tends to use and/or plans to use with respect to performing specific types of surgeries. For instance, the historical use data 108 may include information about the amount of viscoelastic agent an ophthalmic surgeon tends to use when performing a cataract surgery as compared to a corneal transplant surgery.

In these and other embodiments, the historical use data 108 may include information about seasonal trends in surgical supply usage. For example, the historical use data 108 may indicate patterns in surgical supply usage during different times of the year (e.g., during the end of the year when surgical centers see an increase in scheduled surgeries as patients attempt to use insurance benefits such as flexible spending accounts (FSAs) before such benefits expire). In some embodiments, the historical use data 108 may include supplier information. For example, information about supplier performance such as delivery times and/or order fulfillment rates may be included in the historical use data 108.

In these and other embodiments, the inventory management module 102 may be configured to generate one or more predictions of surgical supplies that may be used in upcoming surgeries. Additionally or alternatively, the inventory management module 102 may identify one or more potential surgical supplies for the upcoming surgeries further based on the predictions. In some embodiments, generating the predictions may include identifying one or more surgical supply use patterns and/or trends for the surgical center based on the historical use data 108. For example, generating the prediction may include the inventory management module 102 analyzing the historical use data 108 to identify that a specific surgeon consistently uses 1.5 mL of viscoelastic agent during specific types of surgeries but 1.0 mL of viscoelastic agent during other types of surgeries. In some embodiments, analyzing the historical use data 108 to identify surgical supply use patterns and/or trends at the surgical center may include applying computational techniques to evaluate relationships, detect surgical supply use behaviors, and/or extract information from data associated with one or more previously performed surgeries at the surgical center. For example, the inventory management module 102 may use one or more statistical and/or artificial intelligence (AI) models to analyze patterns in the historical use data 108 with which to generate the predictions of surgical supplies that may be used in upcoming surgeries. For instance, the predictions may be generated using linear regressions, logistic regressions, Bayesian inference models, decision trees, neural networks, and/or any other statistical or AI models. In some embodiments, the inventory management module 102 may generate the predictions of surgical supplies using one or more time-series analysis techniques (e.g., AutoRegressive Integrated Moving Average (ARIMA), Seasonal ARIMA (SARIMA), Long Short-Term Memory (LSTM), etc.). Additionally or alternatively, the inventory management module 102 may predict surgical supplies using one or more natural language processing techniques (e.g., by extracting information from surgery notes, medical records, etc. included in the historical use data 108 that may impact surgical supplies).

In some embodiments, the inventory management module 102 may be configured to generate one or more inventory management actions 110 using the surgery data 104, the inventory data 106, and/or the historical use data 108.

In some embodiments, the inventory management actions 110 may include identifying depleted supplies. For example, the inventory management module 102 may use the inventory data 106 to identify that a particular power of IOLs is not currently stocked at a surgical center.

In some embodiments, the inventory management actions 110 may include generating inventory reports (e.g., real-time inventory reports) that may provide a comprehensive view of available surgical supplies, including quantities, locations, expiration dates, and/or any other information about the surgical supplies. In some embodiments, the inventory management actions 110 may include generating inventory reports that may be customized based on user preferences.

In some embodiments, the inventory management actions 110 may include facilitating the physical retrieval of surgical supplies. For example, the inventory management actions 110 may include activation of location indicators, such as lights that may be illuminated to guide surgical center staff members to the correct storage locations. In some embodiments, location indicators may be color-coded to correspond to specific surgeries and/or users, which may streamline the surgical supply retrieval process.

In some embodiments, the inventory management actions 110 may include automatically updating inventory status designations for surgical supplies, such as transitioning items from “available” to “allocated” when assigned to a particular surgery, and further transitioning to “used” once the surgical supplies have been used in a surgery.

In some embodiments, the inventory management actions 110 may include verifying that specific surgical supplies have been pulled for specific surgeries. For example, one or more additional inventory identifications (e.g., RFID scans) may be performed to verify which surgical supplies were pulled.

In some embodiments, the inventory management actions 110 may include generating notifications about low stock levels. For example, a notification may be generated on an end user device (e.g., via a graphical user interface) of a staff member responsible for ordering IOLs for an ophthalmic surgical center to indicate that the surgical center may not have sufficient multifocal IOLs available based on the surgery data 104 (e.g., there are upcoming surgeries scheduled that will use multifocal IOLs), the inventory data 106 (e.g., the number of multifocal IOLs currently at the surgical center), and/or the historical use data 108 (e.g., the number of additional surgeries using multifocal IOLs in the next month that are expected to be scheduled based on trends from one or more previous years).

In some embodiments, the inventory management actions 110 may include generating alerts for surgical supplies that are approaching their expiration dates. In some embodiments, these alerts may be configured with customizable thresholds (e.g., alerts indicating which surgical supplies expire within the next 30 days, 60 days, 180 days, etc.) to provide advance notice for taking appropriate actions (e.g., actions such as prioritizing the use of soon-to-expire surgical supplies).

In some embodiments, the inventory management actions 110 may include initiating one or more ordering processes. For example, automatically initiating an ordering process for at least one of the depleted surgical supplies for the surgical center may be an inventory management action 110. In these and other embodiments, the ordering process may include one or more of automatically generating a purchase order that includes a particular depleted supply of the depleted surgical supplies based on a particular inventory threshold corresponding to the particular depleted supply. For example, the inventory threshold for glaucoma drainage implants (GDIs) may be set at 40 such that an ordering process including automatically generating a purchase order for GDIs may be initiated in response to fewer than 40 GDIs being stocked at the surgical center. In some embodiments, the inventory management actions 110 may include transmitting one or more purchase orders to a supplier, tracking order status for one or more depleted surgical supplies that were ordered, and/or tracking estimated delivery dates for one or more depleted surgical supplies that were ordered.

In some embodiments, the inventory management actions 110 may include facilitating returns of unused surgical supplies to inventory, which may involve updating inventory records and/or adjusting status designations. In some embodiments, the inventory management actions 110 may include one or more actions related to returning surgical supplies to suppliers.

In some embodiments, the inventory management actions 110 may include one or more order tracking actions. For example, the inventory management actions 110 may include monitoring the status of placed orders and/or estimating delivery dates.

In some embodiments, the inventory management actions 110 may include consignment inventory tracking (e.g., recording usage of consigned surgical supplies, generating reports for billing purposes, facilitating communication with suppliers regarding consignment inventory levels, etc.).

In some embodiments, the inventory management actions 110 may include automated reordering processes triggered by predefined inventory thresholds and/or by the use of surgical supplies during surgeries. For example, the inventory management actions 110 may include generating purchase orders that may be automatically sent to suppliers or queued for approval by an authorized staff member at the surgical center.

In some embodiments, the inventory management actions may include quality control actions, such as recording and/or tracking any issues identified with surgical supplies. In these and other embodiments, the inventory management actions may include one or more actions for facilitating communication with suppliers regarding issues with surgical supplies.

Modifications, additions, or omissions may be made to the system 100 without departing from the scope of the present disclosure. For example, the system 100 may include more or fewer elements depending on the implementation. Further, the system 100 may be configured to perform any number of operations as compared to those explicitly described. In addition, the principles described may be applied to any applicable surgical supply inventory management system and are not necessarily limited to ophthalmic surgical supplies.

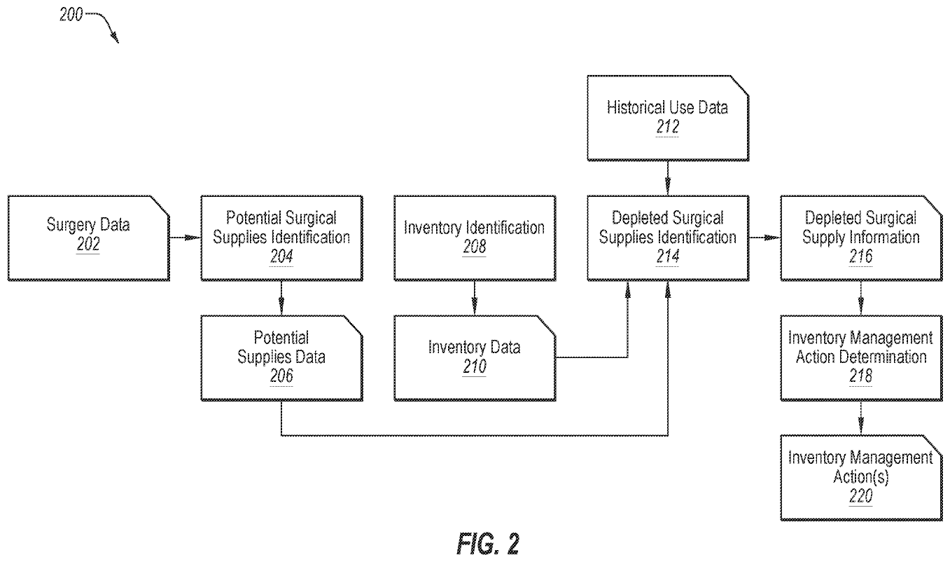

FIG. 2 illustrates an example process 200 that may be performed to generate inventory management actions 220, according to one or more embodiments of the present disclosure. Each operation or block of the process 200 may comprise a computing process that may be performed using any combination of hardware, firmware, and/or software. For example, various functions may be carried out by one or more processors executing instructions stored in memory. The process 200 may be embodied in computer-usable instructions stored on computer storage media in some embodiments. In some embodiments, the process 200 may be provided by a standalone application, a service or hosted service (standalone or in combination with another hosted service), as a microservice via an application programming interface (API), and/or as a plug-in to another product.

By way of example, the process 200 is described with respect to the system 100 described with respect to FIG. 1. For example, the inventory management module 102 of FIG. 1 may be configured to perform one or more of the operations of the process 200 in some embodiments. However, the process 200 may additionally or alternatively be executed by any one system, or any combination of systems, including, but not limited to, those described herein. Further, to ease explanation, the description of the process 200 is given with respect to generating inventory management actions 220 corresponding to surgical supply inventory management of an ophthalmic surgical center, however, such a process may be used for the generation of inventory management actions 220 for any number of other surgical centers.

In some embodiments, the process 200 may generally include generating inventory management actions 220 using surgery data 202 (e.g., the surgery data 104 of FIG. 1), inventory data 210 (e.g., the inventory data 106 of FIG. 1), and/or historical use data 212 (e.g., the historical use data 108 of FIG. 1). In some embodiments, the process 200 may include performing a potential surgical supplies identification 204 using the surgery data 202. In some embodiments, the potential surgical supplies identification 204 may include analyzing the surgery data 202 to identify potential supplies data 206 (e.g., surgical supplies anticipated to be used and/or consumed during surgeries at the surgical center). For example, an ophthalmic surgical center may have 20 surgeries scheduled in which monofocal IOLs are anticipated to be used for a given day such that the potential surgical supplies identification 204 may identify 20 monofocal IOLs as potential surgical supplies included in the potential supplies data 206.

In these or other embodiments, the process 200 may include performing an inventory identification 208. In some embodiments, the inventory identification 208 may include identifying one or more surgical supplies currently stocked in the surgical supply inventory of the surgical center. For example, the inventory identification 208 may include performing an RFID scan using a RFID scanner (e.g., reader) configured in one or more inventory rooms and/or operating rooms of the surgical center to detect RFID tags included on and/or in one or more surgical supplies (e.g., in the surgical supply packaging). In some embodiments, the inventory identification 208 may include scanning individual identification tags (e.g., RFID tags) corresponding to individual surgical supply items at a surgical center to obtain inventory data about the surgical supply inventory at the surgical center. For example, the inventory identification 208 may include scanning an inventory stockroom at the surgical center for individual identification tags corresponding to monofocal IOLs at the surgical center, which may result in the generation of inventory data 210 indicating the number of monofocal IOLs the surgical center currently has in stock (e.g., 11 monofocal IOLs).

In some embodiments, the process 200 may include generating depleted surgical supply information 216 from a depleted surgical supplies identification 214. In some embodiments, the depleted surgical supplies identification 214 may use the potential supplies data 206 from the potential surgical supplies identification 204 and/or the inventory data 210 from the inventory identification 208 to generate the depleted surgical supply information 216. In some embodiments, the depleted surgical supplies identification 214 may include comparing the current inventory of one or more surgical supplies at the surgical center (e.g., based on the inventory data 210) with the potential surgical supplies anticipated to be used at the surgical center (e.g., based on the potential supplies data 206). For example, the comparison may be used to identify surgical supplies that are out of stock or running low at the surgical center. In some embodiments, information about surgical supplies identified as out of stock or running low at the surgical center may be included in the depleted surgical supply information 216.

In these or other embodiments, the depleted surgical supplies identification 214 may use historical use data 212. In some embodiments, the historical use data 212 may be used by the depleted surgical supplies identification 214 to account for one or more historical surgical supply usage trends in identifying depleted surgical supplies. For example, the depleted surgical supplies identification 214 may use the historical use data 212 to include some planned redundancy in surgical supply inventory when identifying depleted surgical supplies. For instance, continuing the examples above, rather than identifying 9 monofocal IOLs as depleted surgical supplies (based on 9 being the difference between the 20 monofocal IOLs identified as potential surgical supplies and the 11 monofocal IOLs identified as in the current inventory), the depleted surgical supplies identification 214 may identify (e.g., account for) 12 monofocal IOLs as depleted surgical supplies based on the historical use data 212 indicating that three additional surgeries using monofocal IOLs tend to be performed any given day at the surgical center).

In some embodiments, the process 200 may include determining one or more inventory management actions 220 based on the depleted surgical supply information 216 using an inventory management action determination 218. In some embodiments, the inventory management action determination 218 may be performed by any suitable system, apparatus, or device. For example, the inventory management action determination 218 may be performed by a computing system and/or an AI system configured to generate inventory management actions 220 based on obtained depleted surgical supply information 216. In some embodiments, the inventory management action determination 218 may analyze the depleted surgical supply information 216 to determine the inventory management actions 220. For example, the inventory management action determination 218 may evaluate the type, quantity, and/or urgency of depleted surgical supplies to determine which inventory management actions 220 may be appropriate. For instance, the inventory management action determination 218 may determine that the inventory management actions 220 should include ordering additional IOLs of a specific power based on the depleted surgical supply information 216 including information regarding that specific power of IOL being low in stock. In some embodiments, the inventory management action determination 218 may consider factors such as reorder thresholds, supplier lead times, upcoming surgery schedules, and/or any other inventory management factors when determining the inventory management actions 220.

In some embodiments, the inventory management actions 220 may be the same or similar to the inventory management actions 110 described with respect to FIG. 1. In some embodiments, the inventory management actions 220 may include ordering the identified depleted surgical supplies included in the depleted surgical supply information 216, updating the inventory data 210 from the inventory identification 208 to reflect ordered surgical supplies, and/or any other actions corresponding to the management of surgical supply inventory at the surgical center. For example, the inventory management actions 220 may include generating a notification on a graphical user interface (GUI) indicating the depleted surgical supplies. In some embodiments, generating the notification may include generating a hyperlink for electronically ordering the depleted surgical supplies.

Modifications, additions, or omissions may be made to the process 200 without departing from the scope of the present disclosure. For example, the process 200 may include more or fewer operations depending on the implementation. In addition, the principles described may be applied to any applicable inventory management process and are not limited to surgical supply inventory management at ophthalmic surgical centers.

FIG. 3 illustrates an example inventory identification system 300 (“system 300”) that may be configured to generate inventory data 316. In some embodiments, the system 300 may generally include an inventory reading system 302 configured to generate an item dataset 314 using supply data 312 corresponding to one or more surgical supply items 308. In these and other embodiments, the system 300 may include an inventory assessment system 318 configured to generate the inventory data 316 using the item dataset 314.

In some embodiments, the surgical supply items 308 may include one or more surgical supplies, such as one or more surgical supplies used in ophthalmic surgeries. For example, the surgical supply items 308 may include eye shields, surgical gloves, surgical masks, surgical gowns, surgical caps, surgical booties, blades and/or scalpels, ophthalmic sutures, ophthalmic cannulas, viscoelastic agents, IOLs, medications, etc.

In some embodiments, each of the surgical supply items 308 may include an identification transponder 310. In some embodiments, the identification transponder 310 may be configured to store and/or transmit supply data 312 corresponding to the surgical supply item 308. In some embodiments, each identification transponder 310 may include a radio-frequency identification (RFID) tag that may be in and/or on each respective surgical supply item 308 (e.g., in and/or on the packing of the surgical supply items 308). In some embodiments, the identification transponder 310 may be configured to transmit the supply data 312 to an inventory reading system 302. For example, the identification transponder 310 may transmit the supply data 312 to the inventory reading system 302 (e.g., in response to the reader 304 performing a read/scan).

In some embodiments, the supply data 312 may include any information that may identify and/or describe the surgical supply items 308. For example, the supply data 312 may include information about the type, size, material, and/or any other characteristic of the surgical supply item 308. In some embodiments, the supply data 312 may include product information such expiration date, lot number, manufacturer information, and/or any other relevant information about the surgical supply items 308. In some embodiments, the supply data 312 may include information about storage requirements for the surgical supply items 308, such as temperature ranges, humidity levels, and/or other environmental conditions. In these and other embodiments, the supply data 312 may include tracking information, such as when and/or where the surgical supply items 308 were manufactured, shipped, received, and/or stored. In some embodiments, the supply data 312 may include status information about the surgical supply items 308 (e.g., availability for use, allocated for use, previously used, etc.).

In some embodiments, the inventory management system 300 may include an inventory reading system 302. In some embodiments, the inventory reading system 302 may include any suitable system, apparatus, or device that may be configured to identify the surgical supply items 308 (e.g., in the surgical supply inventory of a surgical center). For example, in some embodiments, the inventory reading system 302 may include a reader 304 and a control interface 306.

In some embodiments, the reader 304 may include any type of sensor configured to read (e.g., scan) identification transponders 310. In some embodiments, the identification transponder 310 for each of the surgical supply items 308 may include a machine-readable identifier such that the reader 304 may include one or more sensors configured to read machine-readable identifiers including barcodes, high-capacity color barcodes, QR codes, data matrix codes, nearfield communication (NFC) tags, magnetic strips, and/or any other type of machine-readable identifier.

In some embodiments, the reader 304 may include a RFID scanner configured to scan identification transponders 310 that are RFID tags. In these and other embodiments, the identification transponder 310 included in, on, and/or with a surgical supply item 308 may be a RFID tag. In some embodiments, the identification transponder 310 may be configured to communicate supply data 312, which may include information corresponding to the surgical supply item 308, to the inventory reading system 302 (e.g., via the reader 304).

In some embodiments, the reader 304 may be configured to read an identification transponder 310 within a specific location range. Additionally or alternatively, the reader 304 may be configured to read multiple identification transponders 310 simultaneously. For example, the reader 304 may be configured to read identification transponders 310 located anywhere within a particular surgical supply inventory room in a surgical center simultaneously. In some embodiments, the reader 304 may be a handheld device. For example, the reader 304 may be a portable RFID scanner wand that may be carried by a staff member to scan surgical supplies in different areas of a surgical center. In some embodiments, the reader 304 may be a fixed device. For example, the reader 304 may be permanently installed in a surgical supply inventory room or storage area of a surgical center.

In some embodiments, the inventory reading system 302 may include a control interface 306. In some embodiments, the control interface 306 may include any suitable system, apparatus, or device that may be configured to control the reader 304 and/or any other component of the inventory reading system 302. In some embodiments, the control interface 306 may include a computing device. For example, the control interface 306 may include a computer, a tablet, a smartphone, and/or any other suitable computing device. In some embodiments, the control interface 306 may include a user interface. For example, the control interface 306 may include a graphical user interface (GUI) that may allow a user to interact with the inventory reading system 302. In some embodiments, the control interface 306 may be configured to initiate a scan by the reader 304. For example, the control interface 306 may be configured to send a signal to the reader 304 to scan for identification transponders 310.

In some embodiments, the inventory reading system 302 may be configured to generate an item dataset 314 using the supply data 312. For example, the control interface 306 of the inventory reading system 302 may control the reader 304 to scan for the identification transponders 310 of the surgical supply items 308 (e.g., in response to a scheduled scan time being reached, based on a user requesting a scan, etc.). In these and other embodiments, the inventory reading system 302 may obtain the supply data 312 corresponding to the surgical supply items 308 (e.g., surgical supplies located within a certain distance of the reader 304 such as in a surgical supply inventory room). In some embodiments, the inventory reading system 302 may be configured to identify each of the corresponding surgical supply items 308 based on the supply data 312 (e.g., based on unique identifiers included in the supply data 312). In these and other embodiments, the inventory reading system 302 may use the identified surgical supply items 308 to generate and/or organize an item dataset 314 (e.g., a list of the surgical supply items 308).

In some embodiments, the item dataset 314 may include any suitable data about the surgical supply items 308 identified by the inventory reading system 302. In some embodiments, the item dataset 314 may include unique identifiers for each of the surgical supply items 308 based on the supply data 312. Additionally or alternatively, the item dataset 314 may include product characteristics, expiration dates, and/or manufacturing information corresponding to the surgical supply items 308. For example, the inventory reading system 302 may add a timestamp indicating when a scan was performed and/or a location identifier indicating where a scan was performed.

In some embodiments, the control interface 306 of the inventory reading system 302 may be configured to obtain additional information about the surgical supply items 308 based on the supply data 312. For example, in some embodiments, the supply data 312 may contain unique product identifiers of the surgical supply items 308 but may not contain any additional information about the surgical supply items 308. In some embodiments, the control interface 306 may use the unique product identifiers of the surgical supply items 308 identified by the inventory reading system 302 and obtain information about specific surgical supplies corresponding to the unique product identifier (e.g., via the Internet, an internal database, etc.).

In some embodiments, the inventory management system 300 may include an inventory assessment system 318. In some embodiments, the inventory assessment system 318 may be configured to generate inventory data 316 using the item dataset 314. In some embodiments, the inventory assessment system 318 may include any suitable system, apparatus, or device that may be configured to assess the inventory of surgical supplies (e.g., list of the surgical supply items 308) included in the item dataset 314. In some embodiments, the inventory assessment system 318 may include a computing system. For example, the inventory assessment system 318 may include one or more computing devices configured to transform the item dataset 314 into inventory data 316. For example, the item dataset 314 may include a list of unique identifiers from the supply data 312 that are obtained by the inventory reading system 302 such that the inventory assessment system 318 may identify one or more surgical supplies (e.g., products) as the inventory data 316 based on the unique identifiers. In some embodiments, the inventory assessment system 318 and the inventory reading system 302 may be part of the same system and are shown as separate systems in FIG. 3 solely for explanation purposes.

In some embodiments, the inventory data 316 may include any suitable information about the inventory of surgical supplies at a surgical center. In some embodiments, the inventory data 316 may include current stock levels of each surgical supply item 308. In some embodiments, the inventory data 316 may include information about which surgical supplies are expired or are about to expire. In some embodiments, the inventory data 316 may include location information. For example, the inventory data 316 may include information about where different surgical supply items 308 are stored within the surgical center. In some embodiments, the inventory data 316 may be the same or similar to the inventory data 106 described with respect to FIG. 1.

Modifications, additions, or omissions may be made to the system 300 without departing from the scope of the present disclosure. For example, the system 300 may include more or fewer operations depending on the implementation. In addition, the principles described may be applied to any applicable inventory identification and/or inventory assessment process and are not limited to surgical supply inventory management.

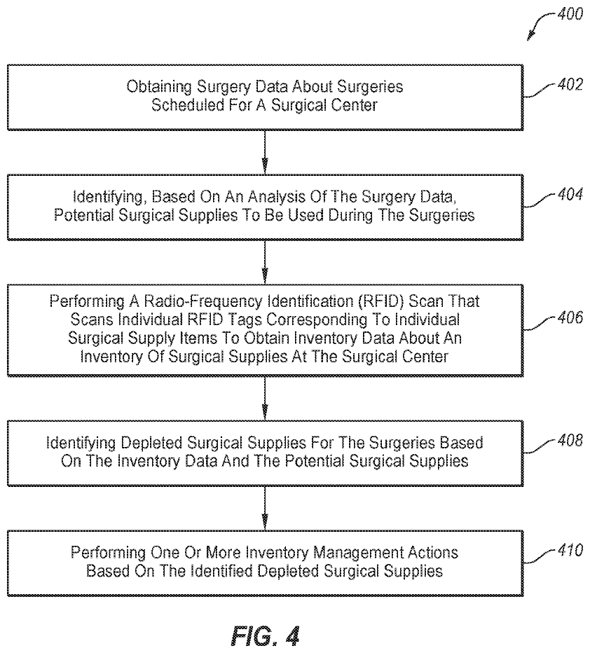

FIG. 4 is a flow diagram illustrating a method 400 of surgical supply inventory management, according to one or more embodiments of the present disclosure. One or more operations of the method 400 may be performed by any suitable system, apparatus, or device, such as the system 100 described with respect to FIG. 1, the inventory management system 202 described with respect to FIG. 2, the inventory reading system 302 and/or the inventory assessment system 318 described with respect to FIG. 3, and/or the computing system 1300 described with respect to FIG. 13 of the present disclosure. In some embodiments, one or more of the operations of the method 400 may be performed by an AI model.

At block 402, surgery data about surgeries scheduled for a surgical center may be obtained. In some embodiments, the surgical center may be an ophthalmic surgical center and the surgeries may be ophthalmic surgeries. In some embodiments, the surgery data may be the same or similar to the surgery data 104 described with respect to FIG. 1 and/or the surgery data 202 described with respect to FIG. 2. In these and other embodiments, the surgery data may include one or more of surgeon information, procedure information, facility information, and/or patient information.

At block 404, potential surgical supplies to be used during the surgeries may be identified based on an analysis of the surgery data. In some embodiments, the potential surgical supplies may be the same or similar to potential surgical supplies identified by the potential surgical supplies identification 204 described with respect to FIG. 2. In some embodiments, the potential surgical supplies may include one or more general surgical supplies (e.g., sterile drapes, gloves, syringes, basic medications, etc.) and/or one or more surgery-specific surgical supplies (e.g., a phacoemulsification handpiece, IOLs, viscoelastic agents, etc. for cataract surgeries).

At block 406, inventory data about an inventory of surgical supplies at the surgical center may be obtained by performing a RFID scan of individual RFID tags corresponding to individual surgical supply items. In some embodiments, the inventory data may be the same or similar to the inventory data 106 described with respect to FIG. 1 and/or the inventory data 316 described with respect to FIG. 3. In these and other embodiments, the inventory data may include one or more of current stock levels of surgical supply items included in the inventory, expiration dates of surgical supply items included in the inventory, unique identifiers for individual surgical supply items, and/or product information for individual surgical supply items. In some embodiments, the RFID scan may be the same or similar to the RFID scan 208 described with respect to FIG. 2. In some embodiments, the RFID scan may be performed by the reader 304 described with respect to FIG. 3. In some embodiments, the individual RFID tags may be the same or similar to the identification transponders 310 described with respect to FIG. 3.

At block 408, depleted surgical supplies for the surgeries may be identified based on the inventory data and the potential surgical supplies. In some embodiments, the depleted surgical supplies may be the same as the depleted surgical supplies identified by the depleted surgical supplies identification 210 described with respect to FIG. 2.

At block 410, one or more inventory management actions may be performed based on the identified depleted surgical supplies. In some embodiments, the inventory management actions may be the same or similar to the inventory management actions 110 described with respect to FIG. 1 and/or the inventory management actions 220 described with respect to FIG. 2. For example, the inventory management actions may include generating a notification (e.g., on a graphical user interface (GUI)) indicating the identified depleted surgical supplies. In these and other embodiments, the notification generated may include a hyperlink for electronically ordering one or more of the depleted surgical supplies.

Modifications, additions, or omissions may be made to the method 400 without departing from the scope of the present disclosure. For example, the operations of method 400 may be implemented in differing order in some instances. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

For example, in some embodiments, the method 400 may include automatically initiating an ordering process for at least one of the depleted surgical supplies for the surgical center. In these and other embodiments, the ordering process may include one or more of automatically generating a purchase order that includes a particular depleted supply of the plurality of deplete surgical supplies based on a particular inventory threshold corresponding to the particular depleted supply, transmitting the purchase order to a supplier, tracking an order status for the particular depleted supply, and/or tracking an estimated delivery date for the particular depleted supply.

In some embodiments, the method 400 may include obtaining historical use data of surgical supplies used in previous surgeries, predicting which surgical supplies will be used during the surgeries (e.g., upcoming scheduled surgeries) based on the historical use data, and/or identifying potential surgical supplies further based on the predicting. In some embodiments, the method 400 may include identifying pulled surgical supplies that are pulled for use during the surgeries and/or updating the inventory data based on the identified pulled surgical supplies. In these and other embodiments, the method 400 may include identifying pulled surgical supplies based on another RFID scan (e.g., a second RFID scan). In these and other embodiments, the method 400 may include automatically initiating a reordering process for pulled surgical supplies.

In some embodiments, the method 400 may include generating a list of supplies to pull from the inventory based on the potential surgical supplies and/or causing activation of a particular location indicator for a particular supply included in the list of supplies based on the particular supply being included in the list of supplies to pull. In these and other embodiments, the particular location indicator may indicate a location of the particular supply in a storage area of the surgical supplies. In some embodiments, the particular location indicator may include a light. In these and other embodiments, the light may be a particular color that corresponds to a particular surgery.

In some embodiments, the method 400 may include assigning a particular item of the potential surgical supplies to a particular surgery and/or transitioning the particular item from an “available” status designation to an “allocated” status designation in the inventory in response to the particular item being assigned to the particular surgery. In these and other embodiments, the method 400 may include transitioning the particular item from the “allocated” status designation to a “used” status designation in the inventory in response to the particular item being used in the particular surgery.

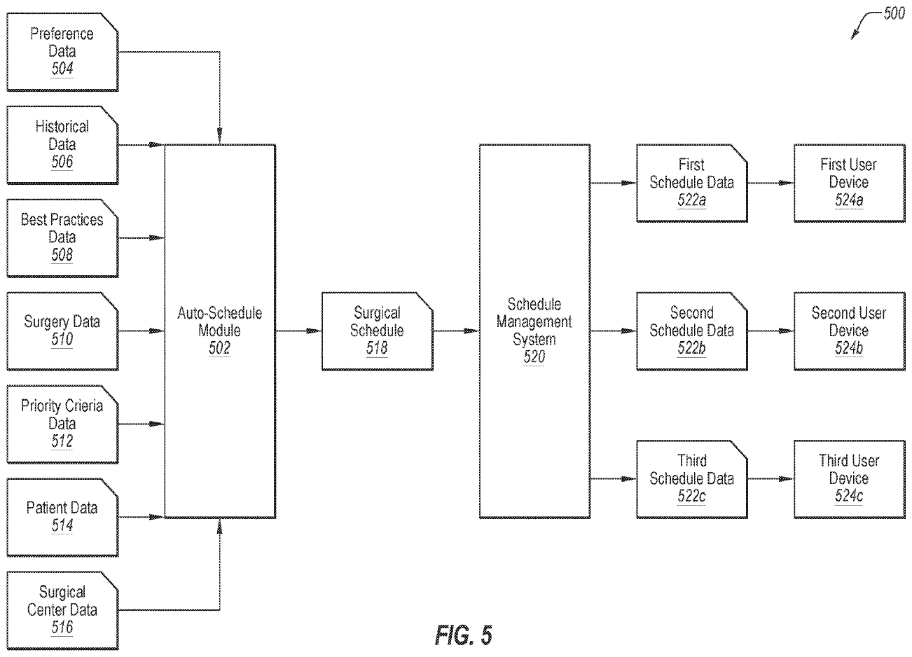

FIG. 5 is a block diagram of an example surgery scheduling system 500 (“system 500”) that may be configured to automatically schedule one or more surgeries at a surgical center according to one or more embodiments of the present disclosure. In general, the system 500 may be configured to perform one or more surgical schedule generation and/or management operations with respect to a surgical schedule corresponding to a surgical center, which may include an ophthalmic surgical center in some embodiments.

In some embodiments, the system 500 may include an auto-schedule module 502 that may be configured to generate a surgical schedule 518 using one or more of: preference data 504, historical data 506, best practices data 508, surgery data 510, priority criteria data 512, patient data 514, or surgical center data 516.

The auto-schedule module 502 may include any suitable system, apparatus, or device that may be configured to automatically schedule surgeries for one or more surgical centers. In some embodiments, the auto-schedule module 502 may include one or more computing systems configured to process and analyze data (e.g., preference data 504, historical data 506, best practices data 508, surgery data 510, priority criteria data 512, patient data 514, surgical center data 516, etc.) from multiple sources (e.g., from surgical supply inventory management systems, electronic medical record systems, etc.). In some embodiments, the auto-schedule module 502 may include one or more different types of devices and/or systems configured to obtain the preference data 504, historical data 506, best practices data 508, surgery data 510, priority criteria data 512, patient data 514, and/or surgical center data 516. For example, the auto-schedule module 502 may include a computing system configured to have data downloaded to, uploaded to, transmitted to, and/or created on the computing system.

For example, the auto-schedule module 502 may be implemented using hardware including one or more processors, central processing units (CPUs), graphics processing units (GPUs), data processing units (DPUs), parallel processing units (PPUs), microprocessors (e.g., to perform or control performance of one or more operations), field-programmable gate arrays (FPGAs), application-specific integrated circuits (ASICs), accelerators (e.g., deep learning accelerators (DLAs)), and/or other processor types. In these and other embodiments, the auto-schedule module 502 may be implemented using a combination of hardware and software.

Additionally or alternatively, the auto-schedule module 502 may include one or more artificial intelligence (AI) systems. In some embodiments, an AI system included in the auto-schedule module 502 may be configured to prioritize surgeries for scheduling and/or to automatically schedule surgeries using one or more AI models. For example, the auto-schedule module 502 may use one or more neural networks, deep learning models, transformer-based models, natural language processing (NLP) models, reinforcement learning models, etc. to generate the surgical schedule 518 using the preference data 504, historical data 506, best practices data 508, surgery data 510, priority criteria data 512, patient data 514, and/or surgical center data 516.

In some embodiments, the auto-schedule module 502 may be configured to obtain preference data 504. In some embodiments, the preference data 504 may include information about the preferences of one or more surgical team members (e.g., surgeons, nurses, anesthesiologists, etc.). For example, the preference data 504 may include information such as preferred surgical techniques, equipment, and/or supplies (e.g., preferred brand of supplies) of the surgical team members. Additionally or alternatively, the preference data 504 may include information about which operating rooms a particular surgeon and/or surgical team prefers to use. In some embodiments, the preference data 504 may include information about preferred working hours, desired breaks between surgeries, and/or any other type of information about the preferences of a surgical team member. In these and other embodiments, the preference data 504 may be obtained from onboarding forms, staff surveys, and/or one or more communication platforms (e.g., via email, instant messaging, file sharing software, etc.).

In some embodiments, the preference data 504 may include patient preferences. For example, the preference data 504 may include information corresponding to specific patients specifying preferences for particular surgeons, appointment times, surgical center locations, etc. In these and other embodiments, the preference data 504 may be obtained from patients using intake forms, patient portals, and/or patient surveys.

In some embodiments, the preference data 504 may include surgical center preferences. For example, the preference data 504 may include information about which days of the week and/or which times of the day the surgical center prefers to schedule surgeries and/or which types of surgeries the surgical center prefers to schedule on certain days or times of day. In these and other embodiments, the preference data 504 may be obtained from one or more surgical center administrators.

Additionally or alternatively, the auto-schedule module 502 may be configured to obtain historical data 506. In some embodiments, the historical data 506 may include information about past surgeries performed at the surgical center. For example, the historical data 506 may include records of surgery durations for different procedure types. In some embodiments, the historical data 506 may help in estimating accurate time slots for upcoming surgeries similar to past surgeries. In some embodiments, the historical data 506 may include information related to surgeon performance metrics, such as average procedure times for specific surgeons performing specific types of surgeries. In some embodiments, the historical data 506 may include information related to previous patients at the surgical center. For example, the historical data 506 may include information about the amount of time it took to check-in a patient, time to prepare a patient for surgery, time post-surgery before the patient was discharged, etc. In some embodiments, the historical data 506 may be obtained from various sources including electronic medical records (EMRs), billing systems, and/or any other source of information about one or more previously scheduled surgeries at the surgical center. In some embodiments, the historical data 506 may be used by the auto-schedule module 502 to identify potential issues (e.g., bottlenecks) in the surgical schedule 518.

In these and other embodiments, the auto-schedule module 502 may be configured to obtain best practices data 508. In some embodiments, the best practices data 508 may include standardized guidelines, protocols, and/or recommended practices used in surgical centers. For example, the best practices data 508 may be obtained from publications and/or websites of organizations such as the American Academy of Ophthalmology (AAO) or the American Society of Ophthalmic Administrators (ASOA). For instance, performing a cataract surgery on a single eye may only take about twenty minutes, however the auto-schedule module 502 may allocate two hours for the full procedure to comport with information included in the best practices data 508 about preoperative steps such as povidone-iodine eye preparation, anesthesia, etc.

In some embodiments, the best practices data 508 may include information about one or more best practices about scheduling within surgical centers. For example, the best practices data 508 may include guidelines from organizations such as the American College of Surgeons (ACS) regarding recommended scheduling patterns for different types of surgeries. In some embodiments, the best practices data 508 may include recommendations for scheduling certain types of surgeries at specific times of day based on clinical outcomes research, such as scheduling more complex cases earlier in the day when surgical teams may be less fatigued. In some embodiments, the best practices data 508 may include information about the best practices for scheduling surgeries based on one or more patient characteristics. For example, the best practices data 508 may indicate that surgeries for older patients may be better to schedule earlier in the day.

In some embodiments, the auto-schedule module 502 may be configured to obtain surgery data 510. In some embodiments, the surgery data 510 may include information about the types of surgeries to be performed, such as a type of ophthalmic surgery (e.g., cataract surgery, corneal transplant, vitreoretinal surgery, etc.). In these and other embodiments, the surgery data 510 may include information about the surgical team assigned to the respective surgeries. For example, the names and roles of one or more surgeons, anesthesiologists, nurses, and/or other staff members involved in individual surgeries may be included in the surgery data 510. In some embodiments, the surgery data 510 may include information about the equipment (e.g., phacoemulsification handpieces, femtosecond lasers, etc.) expected to be used in specific surgeries. In some embodiments, the surgery data 510 may be obtained from various sources within the surgical center. For example, the surgery data 510 may be obtained from electronic medical records at the surgical center. Additionally or alternatively, the surgery data 510 may be obtained from various sources external to the surgical center. For example, the surgery data 510 may include information about a surgery from a referring physician's office. In some embodiments, the surgery data 510 may be the same or similar to the surgery data 104 described with respect to FIG. 1 and/or the surgery data 202 described with respect to FIG. 2.

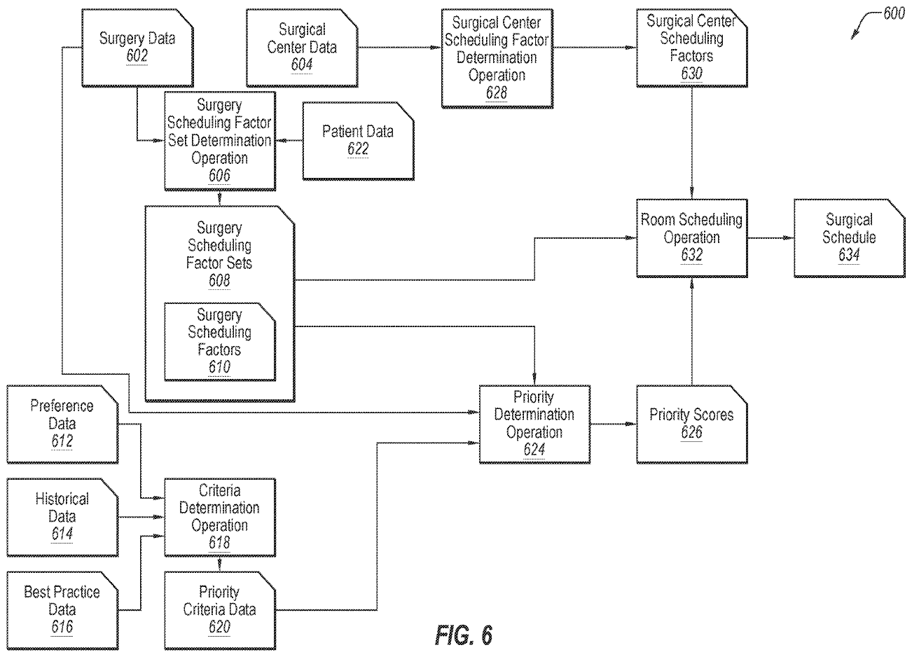

In some embodiments, the auto-schedule module 502 may be configured to obtain priority criteria data 512. In some embodiments, the priority criteria data 512 may include any suitable information about criteria for prioritizing surgeries to be scheduled. In some embodiments, the priority criteria data 512 may include information about medical priority criteria. For example, the priority criteria data 512 may include information about which medical conditions may warrant higher priority for scheduling surgeries (e.g., surgeries for patients with diabetes may be prioritized for scheduling). In some embodiments, the priority criteria data 512 may include information about logistical priority criteria. For example, the priority criteria data 512 may include information about one or more logistical factors (e.g., availability of surgeons, availability of equipment, etc.) that may impact the priority of scheduling surgeries. In some embodiments, the priority criteria data 512 may include information about patient-specific priority criteria. For example, the priority criteria data 512 may include information about one or more patient-specific factors (e.g., age, distance traveled to the surgical center, etc.) that may impact the priority of scheduling surgeries. In some embodiments, the priority criteria data 512 may include data that may correspond to preferences as to when certain types of surgeries may be scheduled. In these and other embodiments, the priority criteria data 512 may be obtained from the preference data 504 and/or from any other source of data corresponding to preferences. In some embodiments, the priority criteria data 512 may be the same or similar to the priority criteria data 620 generated by the criteria determination operation 618 described below with respect to FIG. 6.

In some embodiments, the auto-schedule module 502 may be configured to obtain patient data 514. For example, the patient data 514 may include information about a patient's medical history (e.g., allergies, previous surgeries, current medications, chronic conditions, family medical history, etc.). In some embodiments, the patient data 514 may include information about patient demographics such as patient age, health insurance provider, primary language, occupation, and/or location. Additionally or alternatively, the patient data 514 may include specific information about the patients that may be pertinent to their respective surgeries, such as the IOL type and/or power to be used in an ophthalmic surgery for a specific patient. In some embodiments, the patient data 514 may be the same or similar to the patient data 622 described below with respect to FIG. 6.

In some embodiments, the auto-schedule module 502 may be configured to obtain surgical center data 516. In some embodiments, the surgical center data 516 may include any suitable information about the surgical center where surgeries are to be scheduled. For example, the surgical center data 516 may include information about available operating rooms in the surgical center at a particular time. For example, the surgical center data 516 may include information indicating that three operating rooms are available (e.g., not currently scheduled to be used or are unavailable for maintenance, renovation, etc.) at a given date and time. In some embodiments, the surgical center data 516 may include information about which surgical team members are available at the surgical center for specific dates, days of the week, and/or times. For example, the surgical center data 516 may include information from one or more employee work schedules. In some embodiments, the surgical center data 516 may include information about the surgical equipment available at the surgical center and/or in which operating rooms such surgical equipment may be available. For example, the surgical center data 516 may include information about which femtosecond lasers may be available at an ophthalmic surgical center at a given date and time and/or which operating rooms include such lasers. Additionally or alternatively, the equipment availability information may include information as to whether equipment is portable such that the equipment may be moved between operating rooms or whether equipment is tied to a particular operating room and cannot be moved between surgeries. In some embodiments, surgical equipment availability may correspond to operating rooms that include one or more of an intraoperative aberrometer or a femtosecond laser.

In these and other embodiments, the preference data 504, historical data 506, best practices data 508, surgery data 510, priority criteria data 512, patient data 514, and/or surgical center data 516 may include any suitable type of data. For example, numerical data, character strings that include characters such as letters, symbols, or other characters, a combination of numbers and characters, images, and/or other types of data may be included.

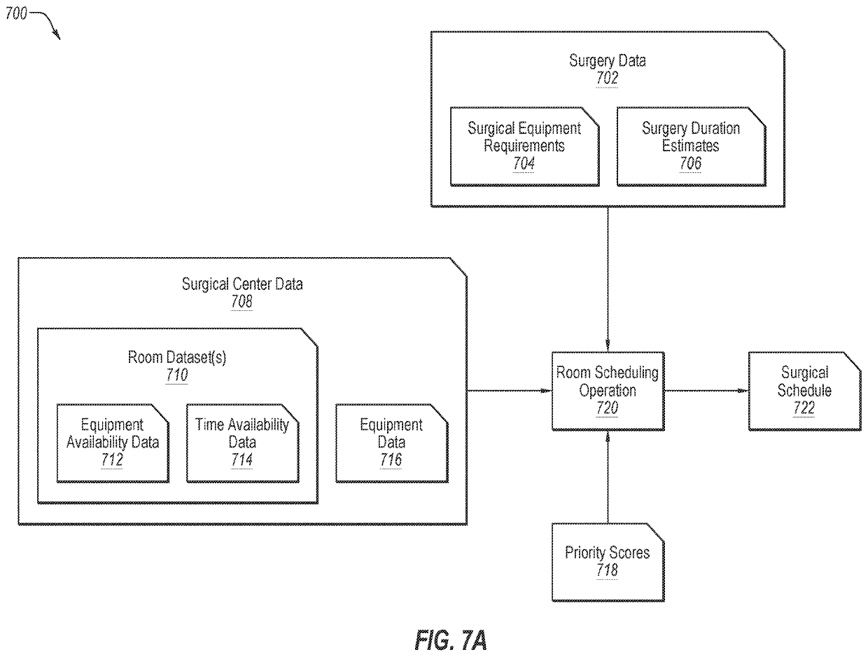

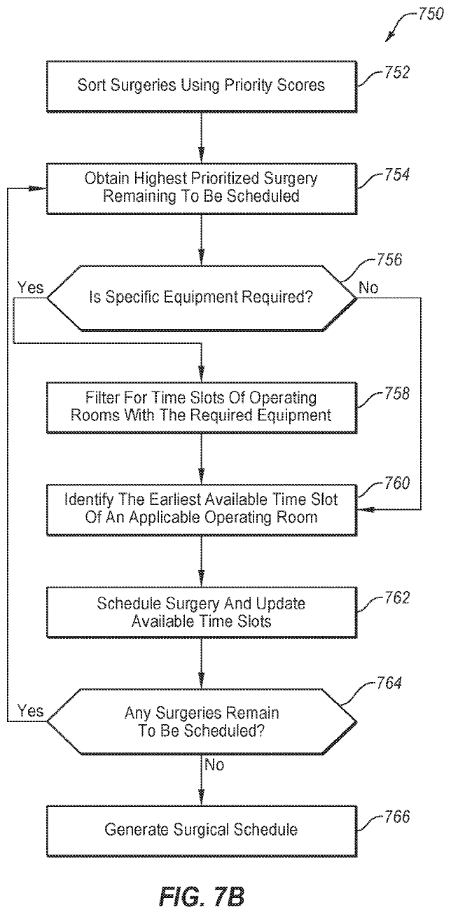

In some embodiments, the auto-schedule module 502 may be configured to generate the surgical schedule 518 using the preference data 504, historical data 506, best practices data 508, surgery data 510, priority criteria data 512, patient data 514, and/or surgical center data 516. For example, in some embodiments, the auto-schedule module 502 may perform one or more operations of the processes described with respect to FIG. 6 and/or FIG. 7 to generate the surgical schedule 518.

In these and other embodiments, generating the surgical schedule 518 may be based on one or more surgical equipment management considerations. In these and other embodiments, the surgical equipment management considerations may be included as at least one scheduling factor included in surgery scheduling factor sets that may be used by the auto-schedule module 502 to generate the surgical schedule 518. For example, the surgical equipment management considerations may include surgical equipment usage information and/or surgical equipment maintenance requirements. In some embodiments, surgical equipment usage information may include frequency of use and/or duration ofuse. For example, the frequency of use and the duration of use may be used by the auto-schedule module 502 in generating the surgical schedule 518 to distribute the use of respective pieces of a type of surgical equipment across various surgeries to be performed at the surgical center. For instance, the auto-schedule module 502 may be configured to generate the surgical schedule 518 such that surgeries requiring a femtosecond laser are assigned to operating rooms in the surgical center with femtosecond lasers based on the frequency of use and/or duration of use of each respective femtosecond laser so as to assist in keeping surgical equipment use (e.g., wear) relatively evenly distributed between all of the femtosecond lasers at the surgical center.

In some embodiments, the surgical schedule 518 may include any suitable information about scheduled surgeries at the surgical center. In some embodiments, the surgical schedule 518 may include information about which surgeries are scheduled on which days and at which times. Additionally or alternatively, the surgical schedule 518 may include information about which surgeons are scheduled to perform which surgeries. In some embodiments, the surgical schedule 518 may include information about which operating rooms are scheduled for which surgeries. Additionally or alternatively, the surgical schedule 518 may include information about which equipment may be used for which surgeries. In some embodiments, the surgical schedule 518 may be presented in any suitable format. For example, the surgical schedule 518 may be a calendar, a list of surgeries, a timeline, a Gantt chart, an interactive dashboard, or any other type of schedule format. In some embodiments, the surgical schedule 518 may be displayed digitally on a graphical user interface (GUI). Additionally or alternatively, the surgical schedule 518 may be printed as one or more physical copies. In some embodiments, the surgical schedule 518 may include details such as patient identifiers, surgery types, assigned surgical teams, estimated durations, scheduled operating rooms, and/or any other details relevant to scheduling surgeries. For example, the surgical schedule 518 may be a digital calendar indicating that on Tuesday, Apr. 22, 2025, the surgeon Dr. Smith is scheduled to perform a LASIK surgery in operating room Suite 142B estimated to last 30 minutes for the patient John Doe.

In some embodiments, user input modifying one or more aspects of the generated surgical schedule 518 may be obtained via a GUI and the surgical schedule 518 may be automatically regenerated based on the user input (e.g., automatically regenerated by the auto-schedule module 502). For example, a patient may indicate using a GUI on a mobile phone device that the patient will be unable to come in for a scheduled surgery such that the surgical schedule 518 may be automatically regenerated to reflect the change.

In some embodiments, a schedule management system 520 may be configured to obtain the surgical schedule 518. In some embodiments, the schedule management system 520 may obtain the surgical schedule 518 from the auto-schedule module 502. For example, in some embodiments the auto-schedule module 502 may automatically transmit the surgical schedule 518 to the schedule management system 520. In these and other embodiments, the schedule management system 520 may obtain the surgical schedule 518 through an application programming interface (API) corresponding to the auto-schedule module 502. In these and other embodiments, the schedule management system 520 may obtain the surgical schedule 518 from a centralized database where multiple surgical schedules may be stored. In some embodiments, the schedule management system 520 may obtain the surgical schedule 518 through a real-time data streaming service that may provide automatic updates in response to one or more changes occurring in the surgical schedule 518.

In some embodiments, the schedule management system 520 may include any suitable system, apparatus, or device that may be configured to manage the surgical schedule 518. The schedule management system 520 may be implemented using hardware including one or more processors, central processing units (CPUs), graphics processing units (GPUs), data processing units (DPUs), parallel processing units (PPUs), microprocessors, field-programmable gate arrays (FPGAs), application-specific integrated circuits (ASICs), accelerators, and/or other processor types. In these and other embodiments, the schedule management system 520 may be implemented using a combination of hardware and software. In some embodiments, the schedule management system 520 may be configured to perform one or more surgery management operations based on the surgical schedule 518.

In some embodiments, the surgery management operations may include notifying a patient, managing inventory of surgical supplies (e.g., ordering surgical supplies), scheduling pre-operative appointments, pulling surgical supplies from a surgical supply inventory for a specific surgery, notifying one or more surgical team members about a scheduled surgery, and/or any other suitable surgery management operation. For example, the surgery management operations may include one or more load balancing operations that may be used to distribute which surgeries are scheduled to use specific pieces of surgical equipment so as to prevent one or more pieces of surgical equipment from being overused.

Additionally or alternatively, the schedule management system 520 may be configured to distribute schedule data 522 (e.g., first schedule data 522a, second schedule data 522b, third schedule data 522c) to one or more user devices 524 (e.g., first user device 524a, second user device 524b, third user device 524c (collectively “user devices 524”)). In these and other embodiments, the first schedule data 522a, the second schedule data 522b, and the third schedule data 522c may include different portions or views of the surgical schedule 518 based on the specific needs and/or roles of the users associated with the respective user devices 524.

In some embodiments, the first user device 524a, the second user device 524b, and/or the third user device 524c may include any suitable electronic devices configured to display and/or interact with the surgical schedule 518. For example, the first user device 524a, the second user device 524b, and/or the third user device 524c may include desktop computers, laptops, tablets, smartphones, dedicated surgical center terminals, wall-mounted displays, and/or any other suitable computing or display devices. In some embodiments, the user devices 524 may be located throughout the surgical center to provide schedule information from the surgical schedule 518 at various locations. For example, the user devices 524 may be positioned at a check-in desk, in pre-operative waiting areas, in operating rooms, in post-operative recovery areas, and/or in surgical supply inventory rooms.

In some embodiments, the first schedule data 522a, the second schedule data 522b, and the third schedule data 522c may each include at least a portion of the surgical schedule 518. For example, the first schedule data 522a may include information about surgeries scheduled for a first surgeon, the second schedule data 522b may include information about surgeries scheduled for a second surgeon, and the third schedule data 522c may include information about surgeries scheduled for a third surgeon. Additionally or alternatively, the first schedule data 522a may include information about surgeries scheduled for a first operating room, the second schedule data 522b may include information about surgeries scheduled for a second operating room, and the third schedule data 522c may include information about surgeries scheduled for a third operating room. Additionally or alternatively, one or more of the first schedule data 522a, the second schedule data 522b, or the third schedule data 522b may include the entire surgical schedule 518.

In these and other embodiments, the first user device 524a, the second user device 524b, and/or the third user device 524c may be configured as check-in devices at the surgical center. For example, a particular user device 524 may be located at a reception area or patient check-in desk and may display one or more portions or views of the surgical schedule 518 for a given day. Such a check-in device may facilitate the check-in process by allowing staff members to quickly access patient information, verify scheduled surgery times, and/or update patient arrival status. For instance, when a patient arrives for a scheduled cataract surgery, a receptionist may use a check-in device (e.g., the first user device 524a) to locate the patient in the surgical schedule 518 and mark the patient as “arrived”.

In some embodiments, the user device 524a, 524b, 524c may be configured to display patient-specific preparation instructions based on the scheduled surgery. For example, when checking in a patient for LASIK surgery, the user device 524a, 524b, 524c may display a reminder to the patient about removing contact lenses prior to the procedure, or when checking in a patient for cataract surgery, the user device 524a, 524b, 524c may display a reminder to a staff member ta the surgical center to administer dilating eye drops to the patient in preparation for the surgery.

Additionally or alternatively, the user device 524a, 524b, 524c may be configured to send automated notifications to patients who have not yet arrived at the surgical center in response to the surgical schedule 518 changing (e.g., due to delays).

In some embodiments, the schedule management system 520 may be integrated with an inventory management system (e.g., the inventory management system 100 described with respect to FIG. 1). In these and other embodiments, the schedule management system 520 may be configured to provide information about scheduled surgeries to the inventory management system, which may use the information about scheduled surgeries to identify potential surgical supplies to be used in those surgeries.

Additionally or alternatively, the inventory management system may provide information about available surgical supplies to the schedule management system 520, which may then generate and/or adjust the surgical schedule 518 based on the surgical supplies availability. For example, in response to the inventory management system identifying that a specific type of IOL needed for a scheduled cataract surgery is not currently in stock at the surgical center, the schedule management system 520 may be configured to flag that surgery in the surgical schedule 518 as potentially requiring rescheduling or alternative supply arrangements. In some embodiments, the schedule management system 520 may be configured to automatically generate notifications to appropriate staff members about potential surgical supply inventory issues that may impact the surgical schedule 518.

In these and other embodiments, the schedule management system 520 may be configured to integrate with other systems at the surgical center, such as electronic medical record (EMR) systems, billing systems, and/or patient communication systems, which may allow for improved information flow between different aspects of surgical center operations and reduced risk of errors. For example, integration of the schedule management system 520 with a patient communication system may allow for automatic sending of appointment reminders, preparation instructions, and/or follow-up information to patients based on the surgical schedule 518.

In some embodiments, the schedule management system 520 may be configured to train an AI model using the surgical schedule 518 (e.g., the generated surgery schedule) and a realized surgery schedule. Additionally or alternatively, the schedule management system 520 may be configured to use an AI model trained using the generated surgery schedule and a realized surgery schedule. In these and other embodiments, the schedule management system 520 may be configured to use the trained AI model to detect one or more scheduling patterns in the realized surgery schedule. In some embodiments, the auto-schedule module 502 may be configured to generate (e.g., regenerate) the surgical schedule 518 further based on the one or more detected scheduling patterns. In these and other embodiments, the scheduling patterns may be detected using one or more of a supervised learning AI model, an unsupervised learning AI model, and/or a reinforcement learning AI model. Additionally or alternatively, the scheduling patterns may be detected using one or more large language models and/or natural language processing.

Modifications, additions, or omissions may be made to the system 500 without departing from the scope of the present disclosure. For example, the system 500 may include more or fewer elements depending on the implementation. For instance, in some embodiments, the preference data 504, the historical data 506, the best practices data 508, the surgery data 510, the priority criteria data 512, the patient data 514, and the surgical center data 516 are delineated in the specific manner described to help with explaining concepts described herein but such delineation is not meant to be limiting. In these and other embodiments, the same or similar data (e.g., overlapping information) may be included in the preference data 504, the historical data 506, the best practices data 508, the surgery data 510, the priority criteria data 512, the patient data 514, and/or the surgical center data 516. For example, the preference data 504 may include information about one or more preferences of a patient that may also be included in the patient data 514. In addition, the principles described may be applied to any applicable surgery scheduling system and are not limited to surgery scheduling at ophthalmic surgical centers.

FIG. 6 illustrates an example process 600 that may be performed to generate a surgical schedule 634, according to one or more embodiments of the present disclosure. Each operation of the process 600 (e.g., surgery scheduling factor set determination operation 606, criteria determination operation 618, priority determination operation 624, surgical center scheduling factor determination operation 628, and/or room scheduling operation 632) may comprise a computing process that may be performed using any combination of hardware, firmware, and/or software. For example, various operations may be carried out by one or more processors executing instructions stored in memory. The process 600 may be embodied in computer-usable instructions stored on computer storage media in some embodiments. In some embodiments, the process 600 may be provided by a standalone application, a service or hosted service (standalone or in combination with another hosted service), as a microservice via an application programming interface (API), and/or as a plug-in to another product.

By way of example, the process 600 is described with respect to the system 500 previously described with respect to FIG. 5. For example, the auto-schedule module 502 of FIG. 5 may be configured to perform one or more of the operations of the process 600 in some embodiments. However, the process 600 may additionally or alternatively be executed by any one system, or any combination of systems, including, but not limited to, those described herein. Further, to ease explanation, the description of the process 600 is given with respect to generating a surgical schedule 634 corresponding to an ophthalmic surgical center, however, such a process may be used for the generation of surgical schedules for any number of other surgical centers (e.g., other types of surgical centers).

In some embodiments, the process 600 may generally include generating the surgical schedule 634 using surgery data 602, surgical center data 604, preference data 612, historical data 614, best practices data 616, and/or patient data 622 by performing one or more of the surgery scheduling factor set determination operation 606, the criteria determination operation 618, the priority determination operation 624, the surgical center scheduling factor determination operation 628, and/or the room scheduling operation 632. In some embodiments, the surgery data 602, the surgical center data 604, the preference data 612, the historical data 614, the best practices 616, the priority criteria data 620, and/or the patient data 622 may be the same or similar as the surgery data 510, the surgical center data 516, the preference data 504, the historical data 506, the best practices data 508, the priority criteria data 512, and/or the patient data 514 as described with respect to FIG. 5, respectively.

In some embodiments, the process 600 may include performing a surgery scheduling factor set determination operation 606 using the surgery data 602 and the patient data 622 to obtain surgery scheduling factor sets 608. For example, the surgery scheduling factor set determination operation 606 may include parsing the surgery data 602. For instance, in some embodiments, the surgery data 602 may include information about multiple surgeries (e.g., all surgeries to be scheduled at a surgical center) such that parsing the surgery data 602 may include determining which information (e.g., surgery scheduling factors 610) corresponds to each specific surgery. Additionally or alternatively, the surgery scheduling factors 610 may include the information from the surgery data 602 that may be considered (e.g., selected to be considered by a user or by an AI model) in scheduling surgeries. In these and other embodiments, the surgery scheduling factors 610 may include information about a patient (e.g., patient health status included in the surgery data 602 and/or information from the patient data 622). In some embodiments, the surgery scheduling factor set determination operation 606 may parse the patient data 622 to determine which information may correspond to a specific surgery to be scheduled at the surgical schedule (e.g., information about the specific patient who will be undergoing the specific surgery).

In some embodiments, the surgery scheduling factor set determination operation 606 may obtain information from the surgery data 602 through various methods to generate the scheduling factor sets 608. The surgery scheduling factor set determination operation 606 may parse the surgery data 602 to extract relevant information for each surgery to be scheduled. For example, the surgery scheduling factor sets 608 may each include multiple surgery scheduling factors 610 such as patient health status, case complexity, surgery type, surgical equipment requirements corresponding to each surgery, and/or surgery duration estimates corresponding to each surgery. Patient health status may include information about one or more preexisting medical conditions (e.g., diabetes, dry eye disease, hypertension, etc.), medication requirements, mobility limitations, and/or recent hospitalization history. Complexity may include a designation of whether the surgery is expected to be “simple” or “complex” based on information such as surgical procedure difficulty ratings, estimated blood loss amounts, and/or potential complications. Surgery type may include information about a type of ophthalmic surgery to be performed. For example, surgery type may include cataract extraction, LASIK, a corneal transplant, vitreoretinal surgery, and/or any other type of ophthalmic surgery. Surgical equipment requirements may include femtosecond lasers, phacoemulsification handpieces, microscopes, and/or any other pieces of equipment used for surgeries. Surgery duration estimates may include information corresponding to the amount of time specific surgeries are expected to take. For example, surgery duration estimates may include information about the expected length of surgeries based on previous amounts of time similar previous surgeries took. In some embodiments, the surgery duration estimates may be based on the surgeon expected to perform the surgery, one or more aspects of the patient the surgery is going to be performed for, preparation and/or post-operative times, and/or any other aspects of the surgery that may impact the time the surgery may last.

By way of example, the surgery scheduling factor set determination operation 606 may identify specific surgery scheduling factors 610 from the surgery data 602 such as Patient ID, Surgery Type, Surgeon ID, Expected Duration, Diabetes Status, Femtosecond Laser requirements, Complexity, and IOL Type as shown below in Table 1.

TABLE 1

| Patient | Surgery | Surgeon | Expected | Diabetes | Femtosecond | | IOL |
|---|---|---|---|---|---|---|---|
| ID | Type | ID | Duration | Status | Laser | Complexity | Type |
| 00001 | LASIK | SMITH | 45 | False | True | Simple | N/A |
| 00002 | Cataract | YE | 30 | False | False | Simple | Toric |
| 00003 | Vitrectomy | MILLER | 45 | True | True | Complex | N/A |

In some embodiments, the surgery scheduling factor set determination operation 606 may utilize pattern recognition techniques to extract structured data from unstructured or semi-structured data in the surgery data 602. For example, the surgery scheduling factor set determination operation 606 may employ natural language processing to identify information from surgical notes or referral documents included in the surgery data 602. Additionally or alternatively, the surgery scheduling factor set determination operation 606 may apply optical character recognition to extract information from scanned documents that may be part of the surgery data 602.

The surgery scheduling factor set determination operation 606 may organize the extracted information into surgery scheduling factor sets 608, with each set corresponding to a specific surgery to be scheduled. For instance, referring to Table 1, the surgery scheduling factor set determination operation 606 may create a surgery scheduling factor set for Patient ID 00001 that includes the factors: LASIK surgery, surgeon Dr. Smith, expected duration of 45 minutes, patient has no diabetes, requires for a femtosecond laser, simple complexity level, and no IOL needed.

In some embodiments, the surgery scheduling factor set determination operation 606 may supplement the information directly available in the surgery data 602 with additional information. For example, once the surgery scheduling factor set determination operation 606 identifies a Patient ID from the surgery data 602, the operation may access patient health records to determine preexisting conditions of the patient that may affect scheduling. For instance, in response to Patient ID 00003 from Table 1 being identified as having diabetes, the surgery scheduling factor set determination operation 606 may further search the patient's health records to determine the type and/or severity of the diabetes, recent A1C levels, or other complications that may influence scheduling priorities or surgical preparation requirements.

In some embodiments, the surgery scheduling factor set determination operation 606 may identify information about surgeon preferences based on the Surgeon ID extracted from the surgery data 602. For example, upon identifying Surgeon YE from Table 1, the surgery scheduling factor set determination operation 606 may look up Dr. Ye's preferred operating room setup, surgical equipment preferences, etc. (e.g., using preference data such as the preference data 504 described with respect to FIG. 5). In some embodiments, one or more individual surgery scheduling factors 610 and/or whole surgery scheduling factor sets 608 may be used in a priority determination operation 624 and/or in a room scheduling operation 632 that are explained below in further detailed.

In some embodiments, the process 600 may include performing a criteria determination operation 618 using the preference data 612, the historical data 614, and/or the best practices data 616 to identify priority criteria data 620. In some embodiments, the priority criteria data 620 identified by the criteria determination operation 618 may include information corresponding to prioritizing specific surgery scheduling factors 610 for scheduling. In these and other embodiments, the criteria determination operation 618 may be configured to extract relevant information from the preference data 612, the historical data 614, and the best practices data 616 through various data processing techniques. For example, the criteria determination operation 618 may include one or more AI data processing techniques such as those including reinforcement learning to learn one or more surgery scheduling factors 610 and/or aspects of surgery scheduling factors 610 to prioritize.

In these and other embodiments, the criteria determination operation 618 may identify priority criteria data 620 (e.g., scheduling priorities) based on the preference data 612. For example, the criteria determination operation 618 may extract information regarding what time of day certain surgeons prefer to perform more complex surgeries. Additionally or alternatively, the criteria determination operation 618 may extract patient preferences from the preference data 612, such as preferred appointment times or days of the week and include such patient preferences into the priority criteria data 620.

In some embodiments, the criteria determination operation 618 may analyze the historical data 614 to identify patterns that may inform scheduling priorities included in the priority criteria data 620. For example, the criteria determination operation 618 may examine past surgical schedules to determine which scheduling approaches have historically resulted in higher operating room utilization rates or fewer delays.

Additionally or alternatively, the criteria determination operation 618 may extract information from the best practices data 616 and incorporate such information into the priority criteria data 620. For example, the criteria determination operation 618 may incorporate recommendations from professional organizations such as the American Academy of Ophthalmology regarding optimal scheduling practices for specific types of ophthalmic surgeries into the priority criteria data 620.

In some embodiments, the criteria determination operation 618 may include assigning weighting factors (e.g., weights) to the different priority criteria. For example, the criteria determination operation 618 may perform a statistical analysis on the preference data 612, the historical data 614, and/or the best practices data 616 to identify correlations between certain surgery scheduling factors 610 and outcomes (e.g., patient satisfaction, surgical center resource utilization, keeping scheduled surgeries on time, etc.) that may be transformed into weighted criteria that may be included in the priority criteria data 620.

In these and other embodiments, the criteria determination operation 618 may organize extracted information from the preference data 612, the historical data 614, and/or the best practices data 616 into different categories of priority criteria to obtain the priority criteria data 620. For example, the priority criteria data 620 may include medical priority criteria (e.g., based on patient condition urgency), logistical priority criteria (e.g., based on surgical center resource availability), and preference-based priority criteria (e.g., based on surgeon and/or patient preferences). In some embodiments, different categories of priority criteria included in the priority criteria data 620 may be assigned different weighting factors based on relative importance as determined by a user (e.g., a member of the surgical center scheduling staff) and/or by an AI model trained to prioritize criteria for surgical scheduling.

In some embodiments, the criteria determination operation 618 may identify conflicts between different sources of priority criteria included in the priority criteria data 620. For example, a surgeon preference from the preference data 612 may conflict with a recommendation from the best practices data 616. In these and other embodiments, the criteria determination operation 618 may apply predefined rules to resolve conflicts, such as prioritizing medical considerations over convenience preferences. For instance, the recommendation to conduct surgeries for elderly patients earlier in the day may be prioritized over the preference of Dr. Young to perform surgeries in the afternoons. In these and other embodiments, weighting factors may be assigned to the individual priority criteria to indicate the relative priority of certain criteria over other criteria.

In some embodiments, the criteria determination operation 618 may generate weighting factors for different priority criteria based on the frequency and/or consistency with which such priority criteria are included across the preference data 612, the historical data 614, and/or the best practices data 616. For example, in response to a particular scheduling consideration appearing frequently in the preference data 612, being supported by patterns in the historical data 614, and/or aligning with recommendations in the best practices data 616, such a scheduling consideration may be assigned a higher weight in the priority criteria data 620.

In some embodiments, the priority criteria data 620 may include any suitable information about criteria for prioritizing surgeries to be scheduled. In some embodiments, the priority criteria data 620 may include information about medical priority criteria. For example, the priority criteria data 620 may include information about which medical conditions may warrant higher priority for scheduling surgeries (e.g., surgeries for patients with diabetes may be prioritized for scheduling). In some embodiments, the priority criteria data 620 may include information about logistical priority criteria. For example, the priority criteria data 620 may include information about one or more logistical factors (e.g., availability of surgeons, availability of equipment, etc.) that may impact the priority of scheduling surgeries. In some embodiments, the priority criteria data 620 may include information about patient-specific priority criteria. For example, the priority criteria data 620 may include information about one or more patient-specific factors (e.g., age, distance traveled to the surgical center, etc.) that may impact the priority of scheduling surgeries. In some embodiments, the priority criteria data 620 may be the same or similar to the priority criteria data 512 described with respect to FIG. 5.

In some embodiments, the process 600 may include performing a priority determination operation 624 using the surgery data 602, one or more individual surgery scheduling factors 610 included in the surgery scheduling factor sets 608, and/or the priority criteria data 620 to determine priority scores 626. In some embodiments, determining the priority scores 626 by performing the priority determination operation 624 may include applying weighting factors to the surgery scheduling factors 610 based on the priority criteria data 620. In these and other embodiments, each surgery scheduling factor such as patient health status, case complexity, surgery type, equipment requirements, etc. may be assigned a specific weighting factor corresponding to the relative importance of one or more specific surgery scheduling factors.

For example, each of the individual priority criteria included in the priority criteria data 620 may receive a weighting factor based on 2\hat{ }n, where n is an integer that increases based on the priority assigned to that individual priority criteria. For instance, surgery type may have an n of 2 while case complexity may have an n of 3 such that the complexity of a surgery may impact the priority scores 626 more than surgery type for prioritizing which surgeries may be scheduled earlier than other surgeries. In these and other embodiments, the weighting factor may be determined by a user and/or by an AI model.

The priority determination operation 624 may include calculating a priority score for each surgery to be scheduled at the surgical center. For example, in some embodiments, the priority score may be determined by multiplying each of the surgery scheduling factors 610 corresponding to a specific surgery by the corresponding weighting factor based on the priority criteria data 620 and summing the weighted values. In these and other embodiments, the priority scores 626 may be used to determine the order in which surgeries are scheduled using the room scheduling operation 632.

In some embodiments, the individual surgery scheduling factors 610 that may be used to determine the priority scores 626 may include patient health status, case complexity, and/or surgery type. Additionally or alternatively, surgical equipment requirements corresponding to each respective surgery and/or surgery duration estimates corresponding to each respective surgery may be used to determine the priority scores 626. For example, the surgical equipment requirements 704 and/or the surgery duration estimates 706 described further below with respect to FIG. 7A may be included in surgery data 602 and/or in one or more of the surgery scheduling factors 610 used to determine the priority scores 626. In some embodiments, the priority determination operation 624 may include combining the surgery data 602, the surgery scheduling factors 610, and/or the priority criteria data 620 using one or more algorithms, data transformation techniques, scoring techniques, and/or ranking techniques. For example, the priority determination operation 624 may include combining the surgery scheduling factors 610, the surgery data 602, and/or the priority criteria data 620 to determine priority scores 626 by applying weighted scoring algorithms, multi-criteria decision analysis techniques, machine learning classification algorithms, regression analysis techniques, etc.

In some embodiments, the priority scores 626 may include a score and/or a rank indicating the priority of each surgery relative to each other surgery of the surgeries to be scheduled at the surgical center. In some embodiments, the surgery scheduling factor sets 608 may provide information about the patient and the surgery such that the priority determination operation 624 may use the priority criteria data 620 to determine which surgeries to schedule prior to scheduling other surgeries. For example, the priority determination operation 624 may include determining a relatively higher priority score 626 for a surgery where the patient has diabetes and the surgery has a high level of complexity. To continue the example, the priority determination operation 624 may determine a relatively lower priority score 626 for a surgery where the patient does not have diabetes and the surgery has a low level of complexity.

In some embodiments, the priority scores 626 may be numerical scores (e.g., 0-100) indicating relative priority of specific surgeries to be scheduled. For example, a surgery with a priority score of 83 may indicate higher priority for scheduling compared to another surgery with a priority score of only 49. Additionally or alternatively, the priority scores 626 may be rankings (e.g., a list of surgeries ranked in order of which surgery should be scheduled first). In some embodiments, the priority scores 626 may be used in the room scheduling operation 632 to generate the surgical schedule 634 described below in further detail.

In some embodiments, the process 600 may include determining one or more surgical center scheduling factors 630 based on the surgical center data 604 using a surgical center scheduling factor determination operation 628. For example, the surgical center scheduling factor determination operation 628 may analyze operating room availability information included in the surgical center data 604 to determine time slots that may be able to accommodate surgeries of various estimated durations. Additionally or alternatively, the surgical center scheduling factor determination operation 628 may evaluate equipment availability information across different operating rooms to determine which operating rooms in the surgical center may be able to accommodate surgeries with various surgical equipment requirements. In these and other embodiments, the surgical center scheduling factors 630 may include any suitable information about the surgical center that may affect surgical scheduling. For example, operating room availability and/or surgical equipment availability may be examples of surgical center scheduling factors 630. In these and other embodiments, the surgical center data 604 may include one or more room datasets that may include equipment availability data and/or time availability data. Additionally or alternatively, the surgical center data 604 may include equipment data. In some embodiments, the surgical center data 604, the room datasets, the equipment availability data, the time availability data, and/or the equipment data may be further described below with respect to FIG. 7A (e.g., the surgical center data 708, the room datasets 710, the equipment availability data 712, the time availability data 714, and/or the equipment data 716).

In some embodiments, the process 600 may include performing a room scheduling operation 632 using the surgery scheduling factor sets 608, the priority scores 626, and/or the surgical center scheduling factors 630. In some embodiments, the surgery scheduling factor sets 608 may include the surgery scheduling factors 610 surgical equipment requirements and/or surgery duration estimates. In these and other embodiments, the surgical equipment requirements may be the same or similar to the surgical equipment requirements described with respect to FIG. 7A. Additionally or alternatively, the surgery duration estimates may be the same or similar to the surgery duration estimates 706 described with respect to FIG. 7A. Each individual surgery scheduling factor set of the surgery scheduling factor sets 608 may correspond to a specific surgery (e.g., with a specific patient, performed by a specific surgeon, etc.). For example, a surgery scheduling factor set may include surgery scheduling factors 610 indicating that a patient has diabetes, that the surgery has a surgical equipment requirement for a femtosecond laser, and that the surgery is estimated to take 60 minutes to perform.

In some embodiments, the room scheduling operation 632 may include sorting surgeries using the priority scores 626. For example, the surgeries may be sorted from lowest to highest priority score, or vice versa. In these and other embodiments, the room scheduling operation 632 may sort the surgeries using the priority scores 626 using one or more sorting algorithms (e.g., quicksort, mergesort, heapsort, etc.).

Additionally or alternatively, the room scheduling operation 632 may include scheduling the surgeries iteratively based on the sorting. For example, the room scheduling operation 632 may include selecting the highest prioritized surgery for scheduling, scheduling that surgery, and then moving to the next highest prioritized unscheduled surgery and scheduling that next highest prioritized unscheduled surgery, etc. The room scheduling operation 632 may be implemented by any suitable system, apparatus, or device that may be configured to scheduler rooms (e.g., operating rooms for surgeries) at the surgical center.

In these and other embodiments, the currently selected surgery may be scheduled using the surgery scheduling factor set 608 (e.g., using at least one surgery scheduling factor such as surgical equipment requirements and/or surgery duration estimates) and/or the surgical center scheduling factors 630 corresponding thereto. For example, the surgical center scheduling factors 630 may include information obtained from the surgical center data 604 such as surgical equipment data, surgical equipment availability data, and time availability data that may be used to determine which surgical equipment may be availability for the surgery, identifying which operating rooms may be available, and/or scheduling the surgery (e.g., designating the specific surgery for the specific patient for a specific operating room at a specific date and time). Additionally or alternatively, the room scheduling operation 632 may be repeated until all surgeries to be scheduled at a surgical center have been scheduled such that a surgical schedule 722 including all of the scheduled surgeries may be generated. In some embodiments, the room scheduling operation 632 may be the same or similar to the room scheduling operation 720 described below with respect to FIG. 7A.

In some embodiments, the surgical schedule 634 may include any suitable arrangement of information about scheduled surgeries at the surgical center. In some embodiments, the surgical schedule 634 may include information about which surgeries are scheduled on which days and at which times. Additionally or alternatively, the surgical schedule 634 may include information about which surgeons are scheduled to perform which surgeries. In some embodiments, the surgical schedule 634 may include information about which operating rooms are scheduled for which surgeries. Additionally or alternatively, the surgical schedule 634 may include information about which equipment may be used for which surgeries. In some embodiments, the surgical schedule 634 may be the same or similar to the surgical schedule 518 described with respect to FIG. 5.

Modifications, additions, or omissions may be made to the process 600 without departing from the scope of the present disclosure. For example, the process 600 may include more or fewer operations depending on the implementation. In addition, the principles described may be applied to any applicable surgery scheduling process and are not limited to surgery scheduling at ophthalmic surgical centers.

FIG. 7A is a block diagram of an example surgical schedule generating process (“process 700”) for generating a surgical schedule 722 using a room scheduling operation 720, according to one or more embodiments of the present disclosure. In general, the process 700 may be configured to perform a room scheduling operation 720 using surgery data 702, surgical center data 708, and/or priority scores 718 to generate the surgical schedule 722.

The room scheduling operation 720 may be implemented by any suitable system, apparatus, or device that may be configured to schedule rooms (e.g., operating rooms for surgeries) at one or more surgical centers. In some embodiments, the room scheduling operation 720 may be the same or similar to the room scheduling operation 632 described with respect to FIG. 6.

In some embodiments, the room scheduling operation 720 may use surgery data 702 to generate the surgical schedule 722. In some embodiments, the surgery data 702 may include surgical equipment requirements 704 and/or surgery duration estimates 706. In some embodiments, the surgical equipment requirements 704 may include information about one or more pieces of surgical equipment expected to be used to perform a specific type of surgery. For example, an intraoperative aberrometry system may be included in the surgical equipment requirements 704 for a cataract surgery because intraoperative aberrometry may be used to provide refractive measurements, allowing for fine-tuning of IOL power and/or placement during such a surgery. As another example, the surgical equipment requirements 704 may include a femtosecond laser system (e.g., for use in LASIK, astigmatic keratotomy, etc.). In some embodiments, the surgery duration estimates 706 may include time lengths that various surgeries are expected to take. In some embodiments, the surgery duration estimates 706 may include information about the expected length of surgeries based on the surgeon expected to perform the surgery, one or more aspects of the patient the surgery is going to be performed for, preparation and/or post-operative times, and/or any other aspects of the surgery that may impact the time the surgery may take. For example, the surgery duration estimates 706 may include expected total time in the operating room including patient preparation, anesthesia administration, the surgical procedure itself, and/or recovery monitoring.

In some embodiments, the room scheduling operation 720 may use surgical center data 708 to generate the surgical schedule 722. In some embodiments, the surgical center data 708 may be the same or similar to the surgical center data 516 described with respect to FIG. 5 and/or the surgical center data 604 described with respect to FIG. 6.

In some embodiments, the surgical center data 708 may include room datasets 710. In some embodiments, the room datasets 710 may include any suitable information about one or more operating rooms at a surgical center. For example, the room datasets 710 may include equipment availability data 712. In some embodiments, the equipment availability data 712 may include information about which equipment is available in which operating rooms at the surgical center. For example, the equipment availability data 712 may indicate that a particular operating room has an intraoperative aberrometry system and/or a femtosecond laser system available for use. Additionally or alternatively, the equipment availability data 712 may include information corresponding to distributing the use of equipment across applicable surgeries that take place at the surgical center. For example, the surgical center may have two different operating rooms that include femtosecond laser systems such that the equipment availability data 712 may include information to assist in balancing the use of each of the two femtosecond laser systems. For instance, the equipment availability data 712 may include information indicating that a first femtosecond laser has been used in four surgeries in the previous week while a second femtosecond laser was used only once in the previous week such that the room scheduling operation 720 may schedule one or more upcoming surgeries requiring a femtosecond laser to be performed in the operating room with the second femtosecond laser.

Additionally or alternatively, the room datasets 710 may include time availability data 714. In some embodiments, the time availability data 714 may include information about when specific operating rooms are available for scheduling surgeries. For example, the time availability data 714 may indicate that a particular operating room is available from 8:00 AM to 3:00 PM on a specific day (e.g., the operating room does not already have a surgery scheduled to take place in the operating room from 8:00 AM to 3:00 PM). In some embodiments, the time availability data 714 may account for one or more breaks (e.g., rest periods, reset periods, etc.). For example, the time availability data 714 may include information corresponding to how much time should be scheduled between two surgeries in a specific operating room (e.g., to allow for instruments and equipment to be sterilized).

In some embodiments, the surgical center data 708 may include equipment data 716. In some embodiments, the equipment data 716 may include any suitable information about surgical equipment at the surgical center. For example, the equipment data 716 may include information about which equipment is operational at the surgical center, which equipment is currently in use, which equipment is scheduled for maintenance, and/or any other information about surgical equipment. In some embodiments, the equipment data 716 may include information about which equipment is required for which types of surgeries. For example, the equipment data 716 may indicate that a phacoemulsification handpiece is required for cataract surgeries.

In some embodiments, the room scheduling operation 720 may use priority scores 718 to generate the surgical schedule 722. In some embodiments, the priority scores 718 may be the same or similar to the priority scores 626 described with respect to FIG. 6. For example, the priority scores 718 may include one or more numerical scores assigned to one or more surgeries.

In some embodiments, the room scheduling operation 720 may generate a surgical schedule 722 using the surgery data 702, the surgical center data 708, and/or the priority scores 718. In some embodiments, the room scheduling operating 720 may compare (e.g., match) the surgical equipment requirements 704 included in the surgery data 702 with one or more operating rooms in the surgical center based on the equipment availability data 712 included in the room datasets 710 of the surgical center data 708. Additionally or alternatively, the room scheduling operation 720 may compare the surgery duration estimates 706 included in the surgery data 702 with one or more operating rooms in the surgical center based on the time availability data 714 included in the room datasets 710 of the surgical center data 708.

In some embodiments, the room scheduling operation 720 may identify surgeries that include a femtosecond laser procedure followed by a main procedure (e.g., a femtosecond laser-assisted cataract surgery (FLACS)). In these and other embodiments, the room scheduling operation 720 may schedule the femtosecond laser procedure and may schedule the main procedure immediately following the femtosecond laser procedure. For example, the room scheduling operation 720 may generate the surgical schedule 722 to have the main procedure scheduled to occur immediately after the femtosecond laser procedure in the same operating room. Alternatively, in some embodiments, the room scheduling operation 720 may generate the surgical schedule 722 to have the main procedure scheduled to occur after the femtosecond laser procedure in a different operating room (e.g., an adjacent operating room without a femtosecond laser to free up the femtosecond laser for another procedure). In some embodiments, scheduling a main procedure of a surgery immediately after a femtosecond laser procedure may improve ophthalmic surgical safety. For example, since a femtosecond laser creates incisions in eyes, having the main procedure immediately after may avoid complications such as capsular contraction, inflammation, infection, etc. In these and other embodiments, immediately may include within 5 minutes, within 10 minutes, or within an hour.

Modifications, additions, or omissions may be made to the process 700 without departing from the scope of the present disclosure. For example, the process 700 may include more or fewer elements depending on the implementation. Further, the process 700 may be configured to perform any number of operations as compared to those explicitly described. In addition, the principles described may be applied to any applicable room scheduling system and are not necessarily limited to surgery scheduling at ophthalmic surgical centers.

FIG. 7B is a block diagram of an example surgical schedule generating process 750 (“process 750”), according to one or more embodiments of the present disclosure. One or more steps of the process 750 may be performed by any suitable system, apparatus, or device such as, for example, the system 500 described with respect to FIG. 5 and/or a system configured to implement the room scheduling operation 632 described with respect to FIG. 6 and/or the room scheduling operation 720 described with respect to FIG. 7A.

At block 752, surgeries may be sorted using priority scores. In some embodiments, the priority scores may be the same or similar to the priority scores 626 described with respect to FIG. 6 and/or the priority scores 718 described with respect to FIG. 7A. For example, the priority scores may be numerical values (e.g., on a scale of 0-100) that may indicate the relative priority of each surgery compared to other surgeries to be scheduled at a surgical center. In some embodiments, sorting surgeries using priority scores may include arranging the surgeries in descending order of priority (e.g., from highest priority score to lowest priority score). Alternatively, sorting surgeries using priority scores may include arranging the surgeries in ascending order of priority (e.g., from lowest priority score to highest priority score). In some embodiments, sorting surgeries using priority scores may include arranging the surgeries using any suitable sorting algorithm (e.g., quicksort, mergesort, heapsort, etc.).

At block 754, the highest prioritized surgery remaining to be scheduled may be obtained. In some embodiments, obtaining the highest prioritized surgery may include selecting the surgery with the highest priority score from the sorted surgeries. In some embodiments, obtaining the highest prioritized surgery may include identifying which surgery has the highest priority score among the surgeries that have not yet been scheduled.

At block 756, a determination may be made regarding whether specific equipment is required for the highest prioritized surgery. In some embodiments, the determination may be made using the surgical equipment requirements 704 included in the surgery data 702 described with respect to FIG. 7A. For example, the determination may be made by checking whether the highest prioritized surgery requires specific equipment such as a femtosecond laser, an intraoperative aberrometry system, and/or any other specialized surgical equipment. In some embodiments, the determination may be made by comparing the surgical equipment requirements 704 for the highest prioritized surgery with the equipment availability data 712 and/or the equipment data 716 described with respect to FIG. 7A. For example, the determination may include identifying which specific equipment is required for the highest prioritized surgery and checking if that equipment is available at the surgical center. In response to specific equipment being required at block 756, the process 750 may proceed to block 758. Alternatively, the process 750 may proceed directly to block 760.

At block 758, time slots of operating rooms with the required equipment may be filtered. In some embodiments, filtering time slots may include identifying which time slots are available for the operating rooms in the surgical center with the required equipment based on the time availability data 714 included in the room datasets 710 of the surgical center data 708 described with respect to FIG. 7A. For example, filtering time slots may include identifying which time slots are available in operating rooms with a femtosecond laser in response to the highest prioritized surgery requiring a femtosecond laser. In some embodiments, the process 750 may proceed to block 760 following block 758.

At block 760, the earliest available time slot of an applicable operating room may be identified. In some embodiments, identifying the earliest available time slot may include determining one or more operating rooms with available time slots long enough to accommodate the highest prioritized surgery based on an estimated surgery duration (e.g., based on the surgery duration estimates 706 included in the surgery data 702 described with respect to FIG. 7A). In some embodiments, identifying the operating room with the earliest available time slot may include checking if any operating rooms are available at a specific time (e.g., at a patient and/or surgical team member requested time). Additionally or alternatively, identifying the earliest available time slot of an applicable operating room may include determining when a specific operating room will become available. In some embodiments, the earliest available time slot may include the earliest available time slot that satisfies one or more surgery scheduling requirements (e.g., patient-specific requirements). For example, the earliest available time slot for a patient with diabetes may be a week later than the earliest generally available time slot because of the patient needing a morning time slot.

At block 762, the highest prioritized surgery may be scheduled for the time slot of the applicable operating room identified and available time slots may be updated. In some embodiments, scheduling the highest prioritized surgery may include assigning the highest prioritized surgery to a specific operating room at a specific date and time. In some embodiments, updating available time slots may include marking the time slot assigned to the highest prioritized surgery as unavailable (e.g., in the time availability data 714 included in the room datasets 710 of the surgical center data 708 described with respect to FIG. 7A). Additionally or alternatively, updating available time slots may include accounting for the estimated duration of the highest prioritized surgery (e.g., based on the surgery duration estimates 706 included in the surgery data 702 described with respect to FIG. 7A). For example, if the highest prioritized surgery is estimated to take two hours, a two-hour time slot scheduled to the highest prioritized surgery may be marked as unavailable.

At block 764, a determination may be made regarding whether any surgeries remain to be scheduled. In some embodiments, the determination may be made by checking that all surgeries with priority scores have been scheduled. For example, the determination may be made by checking for surgeries with priority scores that have not been assigned to an operating room at a specific date and time. In these and other embodiments, the determination may be made by checking that all surgeries meeting certain criteria (e.g., all surgeries for a specific surgeon) have been scheduled.

At block 766, a surgical schedule may be generated if no surgeries remain to be scheduled. In some embodiments, generating the surgical schedule may include compiling information about all scheduled surgeries into a format that may be used by one or more systems, apparatuses, or devices. For example, generating the surgical schedule may include creating a calendar, a list, a timeline, and/or any other suitable representation of the scheduled surgeries. In some embodiments, the surgical schedule may include information about which surgeries are scheduled on which days and at which times. Additionally or alternatively, the surgical schedule may include information about which surgeons are scheduled to perform which surgeries. In some embodiments, the surgical schedule may include information about which operating rooms are scheduled for which surgeries. Additionally or alternatively, the surgical schedule may include information about which equipment may be used for which surgeries. In some embodiments, the surgical schedule may be the same or similar to the surgical schedule 518 described with respect to FIG. 5, the surgical schedule 634 described with respect to FIG. 6, and/or the surgical schedule 722 described with respect to FIG. 7A.

Modifications, additions, or omissions may be made to the method 750 without departing from the scope of the present disclosure. For example, the operations of method 7500 may be implemented in differing order in some instances. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

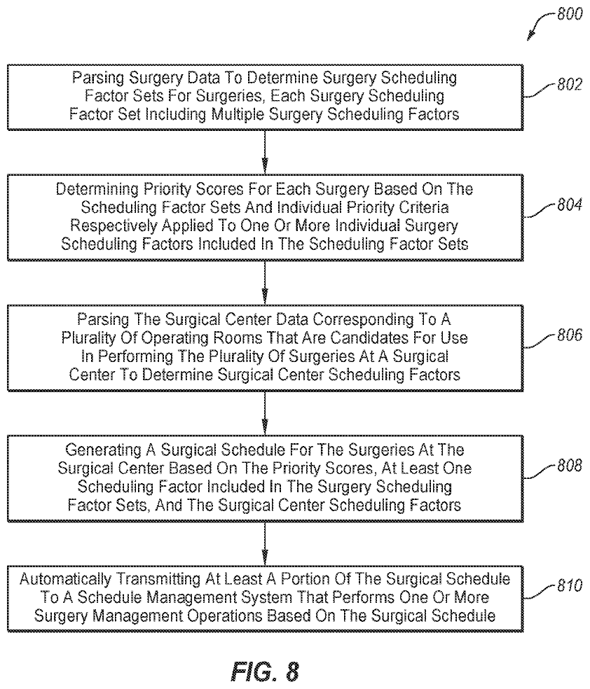

FIG. 8 is a flow diagram illustrating a surgery scheduling method 800 (“method 800”), according to one or more embodiments of the present disclosure. One or more operations of the method 800 may be performed by any suitable system, apparatus, or device, such as the system 500 described with respect to FIG. 5 and/or the computing system 1300 described below with respect to FIG. 13.

At block 802, surgery data and surgical center data may be parsed to determine surgery scheduling factor sets for surgeries. each surgery scheduling factor set including multiple surgery scheduling factors. In some embodiments, parsing the surgery data may include sorting the surgery data into one or more categories of information such as surgical equipment requirements and/or surgery duration estimates. In some embodiments, parsing the surgical center data may include sorting the surgical center data into one or more categories of equipment data, equipment availability data, and/or operating room time availability data. In these and other embodiments, the parsing may be performed by one or more AI models. For example, supervised learning models (e.g., decision trees), neural networks, transformers, and/or any other AI model may be used to determine the surgery scheduling factor sets. In some embodiments, the surgery scheduling factor sets may include one or more individual surgery scheduling factors that may be used to prioritize specific surgeries for scheduling at a surgical center. For example, the surgery scheduling factors may include patient health status, case complexity, surgery type, surgical equipment requirements, and/or surgery duration estimates.

At block 804, priority scores for each surgery may be determined based on the scheduling factor sets and individual priority criteria respectively applied to the one or more individual surgery scheduling factors included in the scheduling factor sets. In some embodiments, the priority criteria may include indications that the surgical center may desire to prioritize one or more aspects of the surgery scheduling factors. For example, the priority criteria may include one or more indications that surgeries for patients who are elderly, for patients with diabetes, performed by a specific surgeon, requiring an intraoperative aberrometry system, etc. may be prioritized (e.g., using one or more weighting factors when determining priority scores). In some embodiments, determining the priority scores for each surgery may include applying the priority criteria to each of the scheduling factor sets. For example, as described above with respect to the priority determination operation 624 in FIG. 6, each individual priority criteria may receive a weighting factor (e.g., a weighting factor that exponentially increases based on the relative priority assigned to that priority criteria) such that the priority scores may be numerical values that may be sorted/ranked to indicate an order of priority in which surgeries may be scheduled.

At block 806, the surgical center data corresponding to operating rooms that are candidates for use in performing the surgeries may be parsed to identify surgical center scheduling factors. In some embodiments, parsing the surgical center data may be used to generate room datasets that may include equipment availability data and/or time availability data corresponding for each operating room in the surgical center.

At block 808 a surgical schedule for the surgeries at the surgical center may be generated based on the priority scores, at least one scheduling factor included in the surgery scheduling factor sets, and/or the surgical center scheduling factors.

At block 810, at least a portion of the surgical schedule may be automatically transmitted to a schedule management system that may perform one or more surgery management operations based on the surgical schedule. In some embodiments, automatically transmitting at least a portion of the surgical schedule may include sending the surgical schedule to the schedule management system. Alternatively, automatically transmitting at least a portion of the surgical schedule may include sending only a subset of the surgical schedule to the schedule management system. For example, automatically transmitting at least a portion of the surgical schedule may include sending only information about surgeries scheduled for a particular day, a particular surgeon, a particular operating room, and/or any other subset of the surgical schedule. In some embodiments, the schedule management system may be configured to perform one or more surgery management operations based on the surgical schedule. For example, the surgery management operations may include notifying a patient about a scheduled surgery, managing inventory of surgical supplies, scheduling pre-operative appointments for a scheduled surgery, and/or any other suitable operation in preparation for or related to managing a scheduled surgery.

In these and other embodiments, the at least one scheduling factor included in the respective surgery scheduling factor sets for generating the surgical schedule may include one or more surgical equipment management considerations. For example, surgical equipment usage information and/or surgical equipment maintenance requirements may be example surgical equipment management considerations. In some embodiments, the surgical equipment usage information may include frequency of use and duration of use such that generating the surgical schedule may include using the frequency of use and the duration of use surgical equipment usage information to distribute surgeries to be scheduled (e.g., spread out which surgeries are scheduled on which pieces of surgical equipment). In these and other embodiments, the surgical equipment usage information may be used to distribute surgeries scheduled to use respective pieces of surgical equipment across multiple pieces of surgical equipment included in a type of surgical equipment. For example, the generating the surgical schedule may include scheduling upcoming surgeries that require a phacoemulsification handpiece across multiple operating rooms with phacoemulsification handpieces rather than scheduling the same operating room with the same phacoemulsification handpiece repeatedly.

Modifications, additions, or omissions may be made to the method 800 without departing from the scope of the present disclosure. For example, the operations of method 800 may be implemented in differing order in some instances. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

For instance,

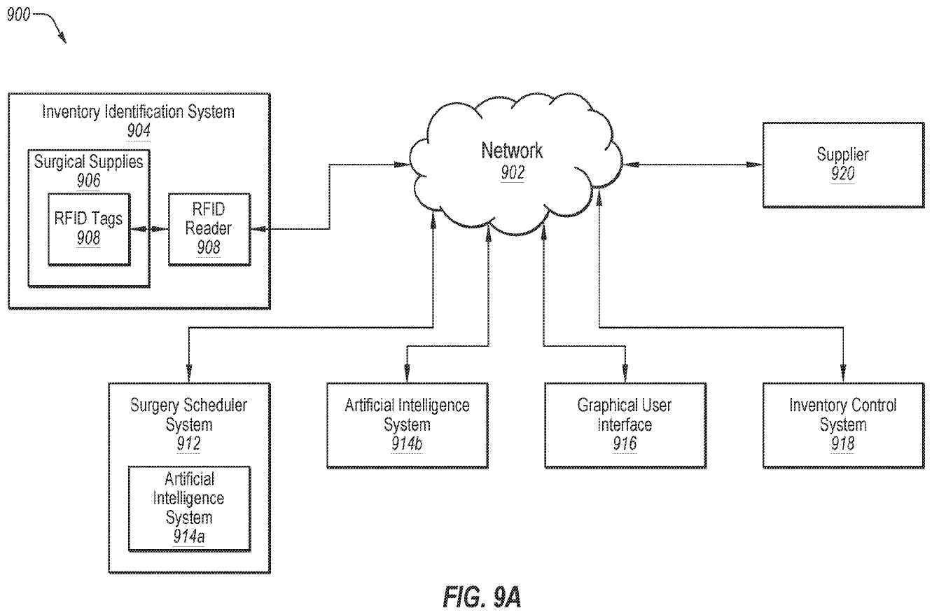

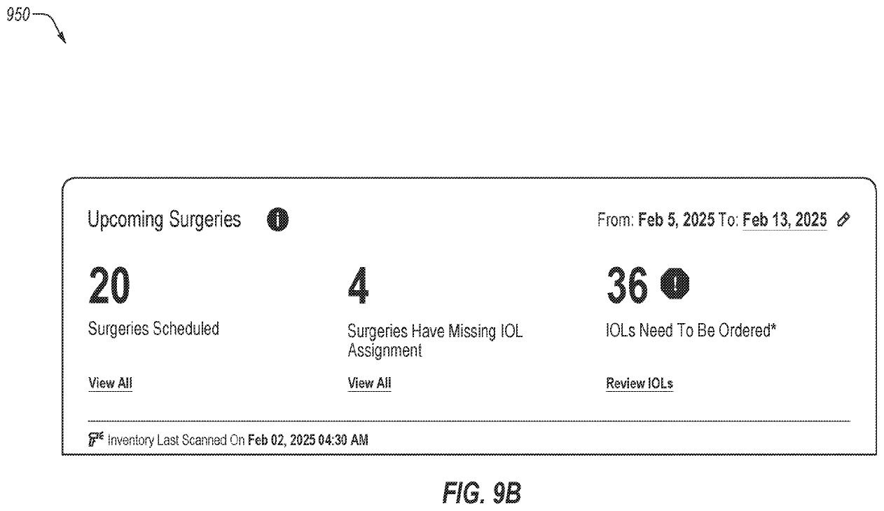

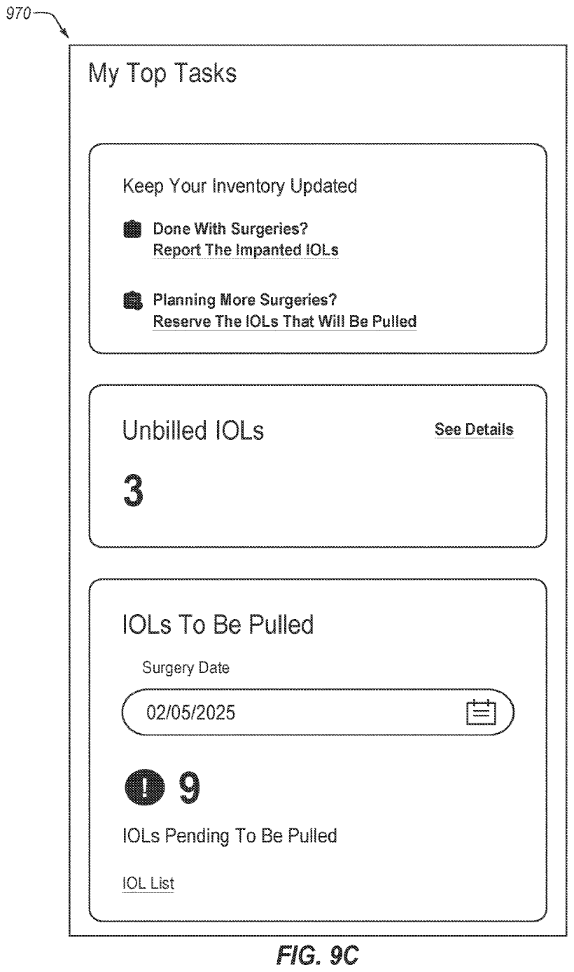

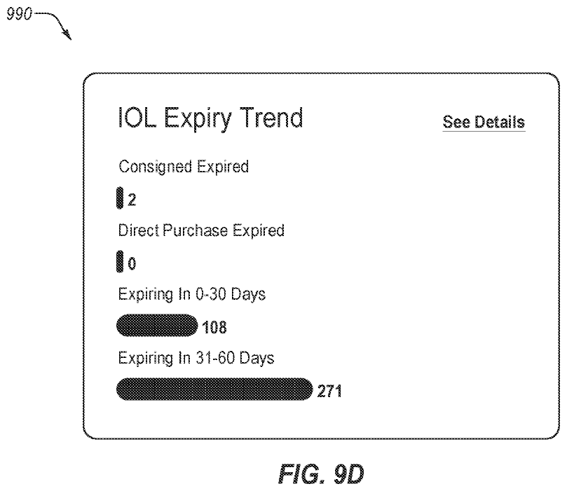

FIG. 9A illustrates an example surgical supply inventory and schedule management system 900 (“system 900”) according to one or more embodiments of the present disclosure. In general, the system 900 may be used by one or more surgical centers (e.g., ophthalmic surgical centers) to manage surgical supply inventory and/or surgery scheduling. In some embodiments, the system 900 may include a network 902 configured to communicatively couple various components of the system 900 (e.g., an inventory identification system 904, a surgery scheduler system 912, one or more artificial intelligence systems 914, a graphical user interface (GUI) 916, an inventory control system 918, and/or a supplier 920).

In some embodiments, the network 902 may include any suitable type of network that may be configured to transmit data between two or more components of the system 900. For example, the network 902 may include one or more of a local area network (LAN), a wide area network (WAN), the Internet, a wireless network, a cellular network, a satellite network, a Bluetooth network, a near-field communication (NFC) network, and/or any other suitable network.

In some embodiments, the network 902 may include one or more secure connections, such as encrypted connections, virtual private networks (VPNs), and/or other secure connections that may provide security for data transmitted over the network 902.

In some embodiments, the system 900 may include an inventory identification system 904. The inventory identification system 904 may be the same or similar to the inventory identification system 300 described with respect to FIG. 3. In some embodiments, the inventory identification system 904 may be configured to identify surgical supplies 906 at a surgical center. For example, the inventory identification system 904 may be configured to scan RFID tags 908 included in and/or on the surgical supplies 906 using an RFID reader 910. In some embodiments, the RFID tags 908 may be the same or similar to the identification transponders 310 described with respect to FIG. 3. In some embodiments, the RFID reader 910 may be the same or similar to the reader 304 described with respect to FIG. 3. For example, the RFID reader 910 may be a device configured to scan the RFID tags 908 and obtain data stored in the RFID tags 908. For instance, the inventory identification system 904 may include a RFID reader 910 that may be installed in an inventory storage room at a surgical center that may include surgical supplies 906 with RFID tags 908 included in the packaging of the surgical supplies 906. To continue the example, the RFID reader 910 may be configured to scan the inventory storage room to identify via the RFID tags 908 that the surgical supplies 906 include specific types of IOLs (e.g., monofocal IOLs, toric IOLs, extended vision IOLs, trifocal IOLs, and/or IOLs of certain powers).

In some embodiments, the system 900 may include a surgery scheduler system 912. In some embodiments, the surgery scheduler system 912 may be configured to schedule surgeries at a surgical center. For example, the surgery scheduler system 912 may be configured to generate a surgical schedule, such as the surgical schedule 518 described with respect to FIG. 5, the surgical schedule 634 described with respect to FIG. 6, and/or the surgical schedule 918 described with respect to FIG. 9. In some embodiments, the surgery scheduler system 912 may be configured to obtain data from the inventory identification system 904, a graphical user interface 916, an inventory control system 918, and/or a supplier 920.

In some embodiments, the surgery scheduler system 912 may include an artificial intelligence system 914a (“AI system 914a”). In some embodiments, the AI system 914a may be configured to analyze data and generate a surgical schedule based on the analyzed data. For example, the AI system 914a may be configured to analyze data such as preference data, historical data, best practices data, surgery data, priority criteria data, patient data, and/or surgical center data to generate a surgical schedule. In some embodiments, the AI system 914a may be configured to use one or more neural networks, decision trees, random forests, support vector machines, and/or any other suitable AI models to generate a surgical schedule.

In some embodiments, the system 900 may include an artificial intelligence system 914b (“AI system 914b”). In some embodiments, the AI system 914b may be configured to analyze data and generate one or more predictions and/or recommendations to any of the various components of the system 900. For example, the AI system 914b may be configured to analyze data such as surgery data, inventory data, and/or historical use data to predict which surgical supplies may be used in upcoming surgeries. In these and other embodiments, the AI system 914b may communicate the prediction about surgical supplies that may be used in upcoming surgeries to the inventory control system 918 via the network 902. In some embodiments, the AI system 914b may assist coordination between one or more of the inventory identification system 904, the surgery scheduler system 912, and the inventory control system 918. For example, the AI system 914b may be configured to recognize and/or learn patterns from the surgery scheduler system 912 about scheduled surgeries at a surgical center, and such patterns may be used by the inventory control system 918 and/or the inventory identification system 904 to manage the surgical supplies 906 based on the scheduled surgeries. In these and other embodiments, the AI system 914a and/or the AI system 914b may be included in the inventory management module 102 described with respect to FIG. 1 and/or in the inventory identification system 300 described with respect to FIG. 3 (e.g., as part of the inventory reading system 302 or the inventory assessment system 318).

In some embodiments, the system 900 may include a graphical user interface 916 (“GUI 916”). In some embodiments, the GUI 916 may be configured to display information to a user and/or receive input from a user. For example, the GUI 916 may be configured to display information about scheduled surgeries, inventory levels, depleted surgical supplies, and/or any other information related to surgical supply inventory and/or schedule management. Additionally or alternatively, the GUI 916 may be configured to receive input from a user related to ordering surgical supplies, scheduling surgeries, and/or any other input related to surgical supply inventory and/or schedule management.

In some embodiments, the GUI 916 may be implemented on any suitable device. For example, the GUI 916 may be implemented on a computer, a tablet, a smartphone, and/or any other suitable device. In some embodiments, the GUI 916 may be implemented as a web-based interface that may be accessed through a web browser. Additionally or alternatively, the GUI 916 may be implemented as a standalone application that may be installed on a device. Specific example GUIs may be given with respect to FIGS. 9B-9D. In some embodiments, the GUI 916 may be used to obtain user input modifying one or more aspects of a generated surgical. In these and other embodiments, the surgical schedule may be automatically regenerated based on the user input obtained via the GUI 916.

FIG. 9B illustrates an example GUI 950. In some embodiments, the GUI 950 may include information regarding one or more scheduled surgeries (e.g., upcoming surgeries) at a surgical center. For example, as illustrated in the time range from Feb. 5, 2025 to Feb. 13, 2025, 20 surgeries may have been scheduled. In some embodiments, the example GUI 950 may include one or more indications (e.g., notifications) corresponding to when the inventory at the surgical center was last scanned. For example, the GUI 950 may indicate what date and/or what time the inventory was last scanned (e.g., by the RFID reader 910). In some embodiments, the GUI 950 may include one or more indications corresponding to surgical supplies (e.g., surgical supplies 906 such as IOLs). For example, the GUI 950 may include an indication that four surgeries are missing IOLs. Additionally or alternatively, the GUI 950 may include an indication that 36 IOLs need to be ordered. In some embodiments, the GUI 950 may include a hyperlink (e.g., the hyperlink “Review IOLs”) to assist in ordering additional IOLs/reviewing purchase orders for IOLs. In some embodiments, the GUI 950 may be configured to automatically update (e.g., make real-time updates) based on an ophthalmic surgical supply inventory management system and/or a surgery auto-scheduler.

FIG. 9C illustrates another example GUI 970. In some embodiments, the GUI 970 may include a notification of surgical supplies to be pulled. For example, for surgeries scheduled on a specific date (e.g., Feb. 5, 2025), the GUI 970 may include the notification that 9 IOLs are pending to be pulled (e.g., from inventory). In some embodiments, the GUI 970 may include hyperlinks allowing a user to interact with one or more surgical inventory and/or surgical scheduling management applications. For example, the GUI 970 may include a hyperlink to allow the user to report surgical supplies that were used in surgeries (e.g., “Report the implanted IOLs” hyperlink). Additionally or alternatively, the GUI 970 may include a hyperlink to allow the user to reserve one or more surgical supplies (e.g., “Reserve the IOLs that will be pulled” hyperlink). In these and other embodiments, the GUI 970 may include a display corresponding to surgical supply inventory billing. For example, in response to one or more IOLs at the surgical center being held on consignment such that once the IOLs have been used in a surgery, then the IOL manufacturer bills for the IOLs, the GUI 970 may indicate how many IOLs are currently unbilled (e.g., have been used in a surgery but not paid for yet).

FIG. 9D illustrates another GUI 990. In some embodiments, the GUI 990 may include a display showing one or more surgical supply inventory expiry trends. For example, IOL expiry trends may be displayed using the GUI 990. For instance, the GUI 990 may display IOL expiry trends in one or more categories such as “Consigned Expired”, “Direct Purchase Expired”, “Expiring in 0-30 days”, and/or “Expiring in 31-60 days”. In some embodiments, the GUI 990 may improve the efficient use of surgical supplies at a surgical center by assisting surgical team members in using surgical supplies that may expire prior to such surgical supplies reaching expiration dates. Although illustrated as separate GUIs, the GUI 950, the GUI 970, and/or the GUI 990, as well as various aspects of the GUI 950, the GUI 970, and/or the GUI 990, may be included in a single GUI (e.g., as separate screens, as part of the same screen, as various application widgets, with one or more hyperlinks to allow a user to interact with multiple aspects of the GUI(s), etc.).

In some embodiments, the system 900 may include an inventory control system 918. In some embodiments, the inventory control system 918 may be configured to manage the inventory of surgical supplies 906 at a surgical center. For example, the inventory control system 918 may be configured to track inventory levels, identify depleted surgical supplies 906, generate purchase orders, and/or perform any other actions related to managing the inventory of surgical supplies 906. In some embodiments, the inventory control system 918 may be configured to communicate with other components of the system 900 via the network 902. For example, the inventory control system 918 may be configured to obtain data from the inventory identification system 904, the surgery scheduler system 912, one or more AI systems 718, the GUI 916, and/or the supplier 920. In some embodiments, the inventory control system 918 may be the same or similar to the system 100 described with respect to FIG. 1.

In some embodiments, the system 900 may include a supplier 920. In some embodiments, the supplier 920 may be configured to provide surgical supplies 906 to a surgical center. For example, the supplier 920 may be a company that manufactures and/or distributes surgical supplies 906 (e.g., IOLs, viscoelastic agents, phacoemulsification handpieces, surgical microscopes, and/or any other surgical supplies 906). In some embodiments, the supplier 920 may be configured to receive purchase orders from the inventory control system 918 and/or to provide information about available surgical supplies 906 to the inventory control system 918.

By way of example, the system 900 may be configured to provide surgical supply inventory and schedule management for an ophthalmic surgical center. For example, the inventory identification system 904 may scan RFID tags 908 of surgical supplies 906 used in ophthalmic surgeries using the RFID reader 910 to identify surgical supplies 906 at the surgical center. In some embodiments, the inventory identification system 904 may transmit data about the identified surgical supplies (e.g., inventory data) to the inventory control system 918 via the network 902. In these and other embodiments, the inventory control system 918 may use the inventory data along with data from the surgery scheduler system 912 about scheduled surgeries (e.g., surgery data) to identify depleted surgical supplies 906. In some embodiments, the inventory control system 918 may generate a notification on the GUI 916 indicating the depleted surgical supplies 906. In some embodiments, the notification may include a hyperlink for electronically ordering the depleted surgical supplies 906 from the supplier 920 (e.g., via the network 902).

### Example Computing System

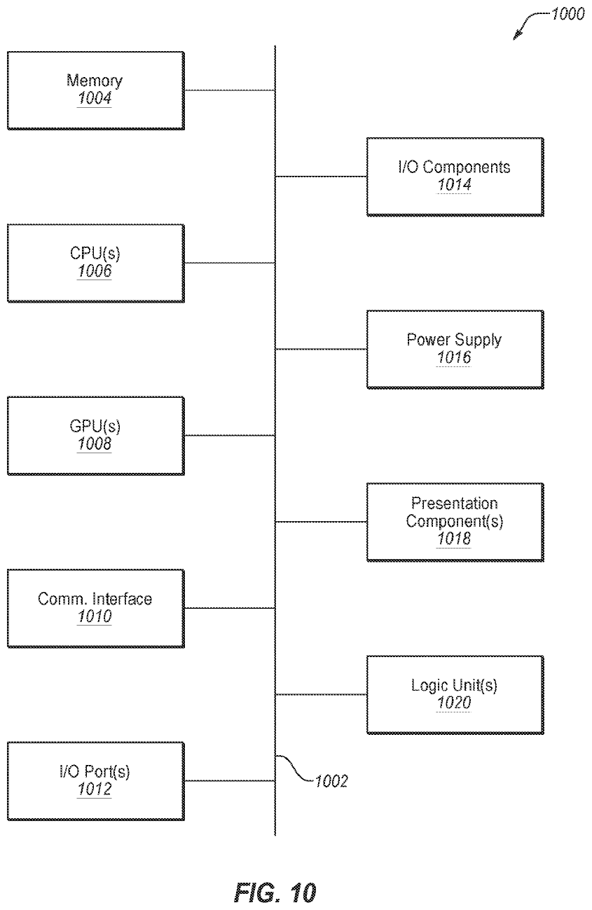

FIG. 10 is a block diagram of an example computing system 1000 suitable for use in implementing some embodiments of the present disclosure. Computing system 1000 may include an interconnect system 1002 that directly or indirectly couples the following devices: memory 1004, one or more central processing units (CPUs) 1006, one or more graphics processing units (GPUs) 1008, a communication interface 1010, I/O ports 1012, input/output components 1014, a power supply 1016, one or more presentation components 1018 (e.g., display(s)), and one or more logic units 1020.

Although the various blocks of FIG. 10 are illustrated as connected via the interconnect system 1002 with lines, this is not intended to be limiting and is for clarity only. For example, in some embodiments, a presentation component 1018, such as a display device, may be considered an I/O component 1014 (e.g., if the display is a touch screen). As another example, the CPUs 1006 and/or GPUs 1008 may include memory (e.g., the memory 1004 may be representative of a storage device in addition to the memory of the GPUs 1008, the CPUs 1006, and/or other components). In other words, the computing system of FIG. 10 is merely illustrative. Distinction is not made between such categories as “workstation,” “server,” “laptop,” “desktop,” “tablet,” “client device,” “mobile device,” “hand-held device,” “game console,” “electronic control unit (ECU),” “virtual reality system,” “augmented reality system,” and/or other device or system types, as all are contemplated within the scope of the computing system of FIG. 10.

The interconnect system 1002 may represent one or more links or busses, such as an address bus, a data bus, a control bus, or a combination thereof. The interconnect system 1002 may include one or more bus or link types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, and/or another type of bus or link. In some embodiments, there are direct connections between components. As an example, the CPU 1006 may be directly connected to the memory 1004. Further, the CPU 1006 may be directly connected to the GPU 1008. Where there is direct, or point-to-point, connection between components, the interconnect system 1002 may include a PCIe link to carry out the connection. In these examples, a PCI bus need not be included in the computing system 1000.

The memory 1004 may include any of a variety of computer-readable media. The computer-readable media may be any available media that may be accessed by the computing system 1000. The computer-readable media may include both volatile and nonvolatile media, and removable and non-removable media. By way of example, and not limitation, the computer-readable media may comprise computer-storage media and communication media.

The computer-storage media may include both volatile and nonvolatile media and/or removable and non-removable media implemented in any method or technology for storage of information such as computer-readable instructions, data structures, program modules, and/or other data types. For example, the memory 1004 may store computer-readable instructions (e.g., that represent a program(s) and/or a program element(s), such as an operating system. Computer-storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information and that may be accessed by computing system 1000. As used herein, computer storage media does not comprise signals per se.

The computer storage media may embody computer-readable instructions, data structures, program modules, and/or other data types in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, the computer storage media may include wired media such as a wired network or direct-wired connection, and wireless media such as acoustic, RF, infrared and other wireless media. Combinations of any of the above should also be included within the scope of computer-readable media.

The CPU(s) 1006 may be configured to execute at least some of the computer-readable instructions to control one or more components of the computing system 1000 to perform one or more of the methods and/or processes described herein. The CPU(s) 1006 may each include one or more cores (e.g., one, two, four, eight, twenty-eight, seventy-two, etc.) that are capable of handling a multitude of software threads simultaneously. The CPU(s) 1006 may include any type of processor, and may include different types of processors depending on the type of computing system 1000 implemented (e.g., processors with fewer cores for mobile devices and processors with more cores for servers). For example, depending on the type of computing system 1000, the processor may be an Advanced RISC Machines (ARM) processor implemented using Reduced Instruction Set Computing (RISC) or an x86 processor implemented using Complex Instruction Set Computing (CISC). The computing system 1000 may include one or more CPUs 1006 in addition to one or more microprocessors or supplementary co-processors, such as math co-processors.

In addition to or alternatively from the CPU(s) 1006, the GPU(s) 1008 may be configured to execute at least some of the computer-readable instructions to control one or more components of the computing system 1000 to perform one or more of the methods and/or processes described herein. One or more of the GPU(s) 1008 may be an integrated GPU (e.g., with one or more of the CPU(s) 1006 and/or one or more of the GPU(s) 1008 may be a discrete GPU. In embodiments, one or more of the GPU(s) 1008 may be a coprocessor of one or more of the CPU(s) 1006. The GPU(s) 1008 may be used by the computing system 1000 to render graphics (e.g., 3D graphics) or perform general purpose computations. For example, the GPU(s) 1008 may be used for General-Purpose computing on GPUs (GPGPU). The GPU(s) 1008 may include hundreds or thousands of cores that are capable of handling hundreds or thousands of software threads simultaneously. The GPU(s) 1008 may generate pixel data for output images in response to rendering commands (e.g., rendering commands from the CPU(s) 1006 received via a host interface). The GPU(s) 1008 may include graphics memory, such as display memory, for storing pixel data or any other suitable data, such as GPGPU data. The display memory may be included as part of the memory 1004. The GPU(s) 1008 may include two or more GPUs operating in parallel (e.g., via a link). The link may directly connect the GPUs (e.g., using NVLINK) or may connect the GPUs through a switch (e.g., using NVSwitch). When combined together, each GPU 1008 may generate pixel data or GPGPU data for different portions of an output or for different outputs (e.g., a first GPU for a first image and a second GPU for a second image). Each GPU may include its own memory, or may share memory with other GPUs.

In addition to or alternatively from the CPU(s) 1006 and/or the GPU(s) 1008, the logic unit(s) 1020 may be configured to execute at least some of the computer-readable instructions to control one or more components of the computing system 1000 to perform one or more of the methods and/or processes described herein. In embodiments, the CPU(s) 1006, the GPU(s) 1008, and/or the logic unit(s) 1020 may discretely or jointly perform any combination of the methods, processes and/or portions thereof. One or more of the logic units 1020 may be part of and/or integrated in one or more of the CPU(s) 1006 and/or the GPU(s) 1008 and/or one or more of the logic units 1020 may be discrete components or otherwise external to the CPU(s) 1006 and/or the GPU(s) 1008. In embodiments, one or more of the logic units 1020 may be a coprocessor of one or more of the CPU(s) 1006 and/or one or more of the GPU(s) 1008.

Examples of the logic unit(s) 1020 include one or more processing cores and/or components thereof, such as Tensor Cores (TCs), Tensor Processing Units (TPUs), Pixel Visual Cores (PVCs), Vision Processing Units (VPUs), Graphics Processing Clusters (GPCs), Texture Processing Clusters (TPCs), Streaming Multiprocessors (SMs), Tree Traversal Units (TTUs), Artificial Intelligence Accelerators (AIAs), Deep Learning Accelerators (DLAs), Arithmetic-Logic Units (ALUs), Application-Specific Integrated Circuits (ASICs), Floating Point Units (FPUs), I/O elements, peripheral component interconnect (PCI) or peripheral component interconnect express (PCIe) elements, and/or the like.

The communication interface 1010 may include one or more receivers, transmitters, and/or transceivers that enable the computing system 1000 to communicate with other computing systems via an electronic communication network, including wired and/or wireless communications. The communication interface 1010 may include components and functionality to enable communication over any of a number of different networks, such as wireless networks (e.g., Wi-Fi, Z-Wave, Bluetooth, Bluetooth LE, ZigBee, etc.), wired networks (e.g., communicating over Ethernet or InfiniBand), low-power wide-area networks (e.g., LoRaWAN, SigFox, etc.), and/or the Internet.

The I/O ports 1012 may enable the computing system 1000 to be logically coupled to other devices including the I/O components 1014, the presentation component(s) 1018, and/or other components, some of which may be built into (e.g., integrated in) the computing system 1000. Illustrative I/O components 1014 include a microphone, mouse, keyboard, joystick, game pad, game controller, satellite dish, scanner, printer, wireless device, etc. The I/O components 1014 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition (as described in more detail below) associated with a display of the computing system 1000. The computing system 1000 may include depth cameras, such as stereoscopic camera systems, infrared camera systems, RGB camera systems, touchscreen technology, and combinations of these, for gesture detection and recognition. Additionally, the computing system 1000 may include accelerometers or gyroscopes (e.g., as part of an inertia measurement unit (IMU)) that enable detection of motion. In some examples, the output of the accelerometers or gyroscopes may be used by the computing system 1000 to render immersive augmented reality or virtual reality.

The power supply 1016 may include a hard-wired power supply, a battery power supply, or a combination thereof. The power supply 1016 may provide power to the computing system 1000 to enable the components of the computing system 1000 to operate.

The presentation component(s) 1018 may include a display (e.g., a monitor, a touch screen, a television screen, a heads-up-display (HUD), other display types, or a combination thereof), speakers, and/or other presentation components. The presentation component(s) 1018 may receive data from other components (e.g., the GPU(s) 1008, the CPU(s) 1006, etc.), and output the data (e.g., as an image, video, sound, etc.).

Modifications, additions, or omissions may be made to FIG. 10 without departing from the scope of the present disclosure. For example, the computing system 1000 may include more or fewer elements depending on the implementation. Further, the computing system 1000 may be configured to perform any number of operations as compared to those explicitly described.

The disclosure may be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to codes that perform particular tasks or implement particular abstract data types. The disclosure may be practiced in a variety of system configurations, including hand-held devices, consumer electronics, general-purpose computers, more specialty computing systems, etc. The disclosure may also be practiced in distributed computing environments where tasks are performed by remote-processing devices that are linked through a communications network.

As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” or “one or more of element A or element B” may include at least one of element A, at least one of element B, or at least one of element A and at least one of element B. Additionally, use of the term “based on” should not be interpreted as “only based on” or “based only on.” Rather, a first element being “based on” a second element includes instances in which the first element is based on the second element but may also be based on one or more additional elements.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

The subject technology of the present disclosure is illustrated, for example, according to various aspects described below. Various examples of aspects of the present disclosure are described as numbered examples (1, 2, 3, etc.) for convenience. These are provided as examples and do not limit the present disclosure. The aspects of the various implementations described herein may be omitted, substituted for aspects of other implementations, or combined with aspects of other implementations unless context dictates otherwise. For example, one or more aspects of example 1 below may be omitted, substituted for one or more aspects of another example (e.g., example 2) or examples, or combined with aspects of another example. The following is a non-limiting summary of some example implementations presented herein.

Example 1. A method comprising:
obtaining surgery data about a plurality of surgeries scheduled for a surgical center;
identifying, based on an analysis of the surgery data, a plurality of potential surgical supplies to be used during the plurality of surgeries;
performing a radio-frequency identification (RFID) scan that scans individual RFID tags corresponding to individual surgical supply items to obtain inventory data about an inventory of surgical supplies at the surgical center;
identifying a plurality of depleted surgical supplies for the plurality of surgeries based on the inventory data and the plurality of potential surgical supplies; and
performing one or more inventory management actions based on the identified plurality of depleted surgical supplies.

Example 2. The method of Example 1, wherein the one or more inventory management actions include automatically initiating an ordering process for at least one of the plurality of depleted surgical supplies for the surgical center.

Example 3. The method of Example 2, wherein the ordering process includes one or more of:
automatically generating a purchase order that includes a particular depleted supply of the plurality of depleted surgical supplies based on a particular inventory threshold corresponding to the particular depleted supply;
transmitting the purchase order to a supplier;
tracking an order status for the particular depleted supply; or
tracking an estimated delivery date for the particular depleted supply.

Example 4. The method of Example 1, further comprising:
obtaining historical use data of surgical supplies used in a plurality of previous surgeries;
predicting which surgical supplies will be used during the plurality of surgeries based on the historical use data; and
identifying the plurality of potential surgical supplies further based on the predicting.

Example 5. The method of Example 1, further comprising:
identifying a plurality of pulled surgical supplies that are pulled for use during the plurality of surgeries; and
updating the inventory data based on the identified plurality of pulled surgical supplies.

Example 6. The method of Example 5, wherein the plurality of pulled surgical supplies are identified based on another RFID scan.

Example 7. The method of Example 5, further comprising automatically initiating a reordering process for the plurality of pulled surgical supplies.

Example 8. The method of Example 1, further comprising:
generating a list of supplies to pull from the inventory based on the plurality of potential surgical supplies; and
causing activation of a particular location indicator for a particular supply included in the list of supplies based on the particular supply being included in the list of supplies to pull, the
particular location indicator indicating a location of the particular supply in a storage area of the surgical supplies.

Example 9. The method of Example 8, wherein the particular location indicator includes a light.

Example 10. The method of Example 9, wherein the light is a particular color that corresponds to a particular user.

Example 11. The method of Example 9, wherein the light is a particular color that corresponds to a particular surgery.

Example 12. The method of Example 1, wherein the surgery data includes one or more of:
surgeon information;
procedure information;
facility information; or
patient information.

Example 13. The method of Example 1, wherein the inventory data includes one or more of:
current stock levels of surgical supply items included in the inventory;
expiration dates of surgical supply items included in the inventory;
unique identifiers for individual surgical supply items; or
product information for individual surgical supply items.

Example 14. The method of Example 1, further comprising:
assigning a particular item of the plurality of potential surgical supplies to a particular surgery; and
transitioning the particular item from an “available” status designation to an “allocated” status designation in the inventory in response to the particular item being assigned to the particular surgery.

Example 15. The method of Example 14, further comprising transitioning the particular item from the “allocated” status designation to a “used” status designation in the inventory in response to the particular item being used in the particular surgery.

Example 16. The method of Example 1, wherein the one or more inventory management actions include generating a notification on a graphical user interface indicating the plurality of depleted surgical supplies, the generating of the notification including generating a hyperlink for electronically ordering the plurality of depleted surgical supplies.

Example 17. A system configured to perform the method of any of Examples 1-16.

Example 18. A method comprising:
parsing surgery data to determine a plurality of surgery scheduling factor sets each corresponding to a respective surgery of a plurality of surgeries, each surgery scheduling factor set of the plurality of surgery scheduling factor sets including a plurality of surgery scheduling factors;
determining a plurality of priority scores with respect to the plurality of surgeries based on the plurality of scheduling factor sets and based on individual priority criteria respectively applied to one or more individual surgery scheduling factors included in the scheduling factor sets, each priority score of the plurality of priority scores corresponding to a respective surgery of the plurality of surgeries and being based on respective weighting factors applied to the one or more individual scheduling factors in which the respective weighting factors are based on the individual priority criteria corresponding to the one or more individual scheduling factors;
parsing surgical center data corresponding to a plurality of operating rooms that are candidates for use in performing the plurality of surgeries at a surgical center to determine a plurality of surgical center scheduling factors, the surgical center scheduling factors including one or more of:
   respective surgical equipment availability corresponding to each operating room of the plurality of operating rooms, and
   respective operating room time availability corresponding to time slots that each operating room of the plurality of operating rooms is available for surgery;
generating a surgical schedule for the plurality of surgeries at the surgical center based on:
   the plurality of priority scores,
   at least one scheduling factor included in the plurality of surgery scheduling factor sets, and
   the surgical center scheduling factors; and
automatically transmitting at least a portion of the surgical schedule to a schedule management system that performs one or more surgery management operations based on the surgical schedule.

Example 19. The method of Example 18, wherein the plurality of surgery scheduling factors include one or more of:
patient health status,
case complexity,
surgery type,
surgical equipment requirements corresponding to the respective surgeries, or
surgery duration estimates corresponding to the respective surgeries.

Example 20. The method of Example 18, wherein the one or more individual surgery scheduling factors used to determine the plurality of priority scores include one or more of: patient health status, case complexity, or surgery type.

Example 21. The method of Example 20, wherein the one or more individual surgery scheduling factors used to determine the plurality of priority scores further include one or more of:
surgical equipment requirements corresponding to the respective surgeries, or
surgery duration estimates corresponding to the respective surgeries.

Example 22. The method of Example 18, wherein the at least one scheduling factor included in the respective surgery scheduling factor sets for generating the surgical schedule includes one or more of:
surgical equipment requirements corresponding to the respective surgeries, or
surgery duration estimates corresponding to the respective surgeries.

Example 23. The method of Example 18, wherein the respective surgical equipment availability corresponds to operating rooms that include one or more of: an intraoperative aberrometer or a femtosecond laser.

Example 24. The method of Example 18, further comprising:
training an artificial intelligence model using the generated surgery schedule and a realized surgery schedule;
using the trained artificial intelligence model to detect one or more scheduling patterns in the realized surgery schedule; and
generating the surgery schedule further based on the one or more detected scheduling patterns.

Example 25. The method of Example 18, wherein generating the surgical schedule is further based on:
identifying one or more surgeries including a femtosecond laser procedure followed by a main procedure;
scheduling the femtosecond laser procedure for each of the one or more identified surgeries; and
scheduling the main procedure immediately following the femtosecond laser procedure for each of the one or more identified surgeries.

Example 26. The method of Example 18, further comprising:
obtaining, via a graphical user interface, user input modifying one or more aspects of the generated surgical schedule; and
automatically regenerating the surgical schedule based on the user input.

Example 27. The method of Example 18, wherein the one or more surgery management operations include one or more of: notifying a patient, managing inventory of surgical supplies, or scheduling pre-operative appointments.

Example 28. The method of Example 18, wherein the at least one scheduling factor included in the respective surgery scheduling factor sets for generating the surgical schedule includes one or more surgical equipment management considerations.

Example 29. The method of Example 28, wherein the one or more surgical equipment management considerations include one or more of: surgical equipment usage information or surgical equipment maintenance requirements.

Example 30. The method of Example 29, wherein the surgical equipment usage information includes frequency of equipment use and duration of equipment use, and
wherein generating the surgical schedule includes using the frequency of equipment use and the duration of equipment use to distribute surgeries scheduled to use respective pieces of surgical equipment across a plurality of pieces of surgical equipment of a type of surgical equipment.

Example 31. A system configured to perform the method of any of Examples 18-30.

What is claimed is:

1. A method comprising:
obtaining surgery data about a plurality of surgeries scheduled for a surgical center;
identifying, based on an analysis of the surgery data, a plurality of potential surgical supplies to be used during the plurality of surgeries;
performing a radio-frequency identification (RFID) scan that scans individual RFID tags corresponding to individual surgical supply items to obtain inventory data about an inventory of surgical supplies at the surgical center;
identifying a plurality of depleted surgical supplies for the plurality of surgeries based on the inventory data and the plurality of potential surgical supplies; and
performing one or more inventory management actions based on the identified plurality of depleted surgical supplies.

2. The method of claim 1, wherein the one or more inventory management actions include automatically initiating an ordering process for at least one of the plurality of depleted surgical supplies for the surgical center.

3. The method of claim 2, wherein the ordering process includes one or more of:
automatically generating a purchase order that includes a particular depleted supply of the plurality of depleted surgical supplies based on a particular inventory threshold corresponding to the particular depleted supply;
transmitting the purchase order to a supplier;
tracking an order status for the particular depleted supply; or
tracking an estimated delivery date for the particular depleted supply.

4. The method of claim 1, further comprising:
obtaining historical use data of surgical supplies used in a plurality of previous surgeries;
predicting which surgical supplies will be used during the plurality of surgeries based on the historical use data; and
identifying the plurality of potential surgical supplies further based on the predicting.

5. The method of claim 1, further comprising:
identifying a plurality of pulled surgical supplies that are pulled for use during the plurality of surgeries; and
updating the inventory data based on the identified plurality of pulled surgical supplies.

6. The method of claim 5, wherein the plurality of pulled surgical supplies are identified based on another RFID scan.

7. The method of claim 5, further comprising automatically initiating a reordering process for the plurality of pulled surgical supplies.

8. The method of claim 1, further comprising:
generating a list of supplies to pull from the inventory based on the plurality of potential surgical supplies; and
causing activation of a particular location indicator for a particular supply included in the list of supplies based on the particular supply being included in the list of supplies to pull, the particular location indicator indicating a location of the particular supply in a storage area of the surgical supplies.

9. The method of claim 8, wherein the particular location indicator includes a light.

10. The method of claim 9, wherein the light is a particular color that corresponds to a particular user.

11. The method of claim 9, wherein the light is a particular color that corresponds to a particular surgery.

12. The method of claim 1, wherein the surgery data includes one or more of:
surgeon information;
procedure information;
facility information; or
patient information.

13. The method of claim 1, wherein the inventory data includes one or more of:
current stock levels of surgical supply items included in the inventory;
expiration dates of surgical supply items included in the inventory;
unique identifiers for individual surgical supply items; or
product information for individual surgical supply items.

14. The method of claim 1, further comprising:
assigning a particular item of the plurality of potential surgical supplies to a particular surgery; and
transitioning the particular item from an “available” status designation to an “allocated” status designation in the inventory in response to the particular item being assigned to the particular surgery.

15. The method of claim 14, further comprising transitioning the particular item from the “allocated” status designation to a “used” status designation in the inventory in response to the particular item being used in the particular surgery.

16. The method of claim 1, wherein the one or more inventory management actions include generating a notification on a graphical user interface indicating the plurality of depleted surgical supplies, the generating of the notification including generating a hyperlink for electronically ordering the plurality of depleted surgical supplies.

17. A surgical supply inventory management system comprising:
a plurality of surgical supply packages corresponding to a plurality of surgical supplies and including a plurality of radio-frequency identification (RFID) transponders associated therewith, each respective surgical supply package including a respective RFID transponder corresponding to a respective surgical supply item of the plurality of surgical supplies and included in the respective surgical supply package, the respective RFID transponder being configured to perform operations comprising:
storing item data corresponding to information about the respective surgical supply item; and
transmitting the item data in response to an RFID scan;
an inventory identification system including:
an RFID reader configured to perform the RFID scan; and
a control interface configured to perform operations comprising:
controlling the RFID scan performed by the RFID reader;
aggregating item data obtained during the RFID scan into an item data set that includes item data corresponding to different surgical supply items of the plurality of surgical supplies; and
communicating the item data set;
an inventory control system configured to perform operations comprising:
receiving the item data set as communicated by the inventory identification system;
generating inventory data for a surgical center at which the surgical supply packages are located, the inventory data indicating an inventory of surgical supply items at the surgical center;
obtaining surgery data about a plurality of surgeries scheduled for the surgical center;
identifying, based on an analysis of the surgery data, a plurality of potential surgical supplies to be used during the plurality of surgeries;
identifying a plurality of depleted surgical supplies for the plurality of surgeries based on the inventory data and the plurality of potential surgical supplies; and
performing one or more inventory management actions based on the identified plurality of depleted surgical supplies.

18. The surgical supply inventory management system of claim 17, wherein the one or more inventory management actions include one or more of automatically initiating an ordering process for the at least one of the plurality of depleted surgical supplies and generating a notification on a graphical user interface indicating the plurality of depleted surgical supplies, the generating of the notification including generating a hyperlink for electronically ordering the plurality of depleted surgical supplies.

19. The surgical supply inventory management system of claim 18, wherein the ordering process includes one or more of:
automatically generating a purchase order that includes a particular depleted supply of the plurality of depleted surgical supplies based on a particular inventory threshold corresponding to the particular depleted supply;
transmitting the purchase order to a supplier;
tracking an order status for the particular depleted supply; or
tracking an estimated delivery date for the particular depleted supply.

20. The surgical supply inventory management system of claim 17, wherein the inventory control system is further configured to:
obtain historical use data of surgical supplies used in a plurality of previous surgeries;
predict which surgical supplies will be used during the plurality of surgeries based on the historical use data; and
identify the plurality of potential surgical supplies further based on the predicting.

21. The surgical supply inventory management system of claim 17, wherein the inventory control system is further configured to:
identify a plurality of pulled surgical supplies that are pulled for use during the plurality of surgeries; and
update the inventory data based on the plurality of pulled surgical supplies.

22. The surgical supply inventory management system of claim 21, wherein the plurality of pulled surgical supplies are identified based on another RFID scan.

23. The surgical supply inventory management system of claim 21, wherein the inventory control system is further configured to automatically initiate a reordering process for the plurality of pulled surgical supplies.

24. The surgical supply inventory management system of claim 17, wherein the inventory control system is further configured to:
generate a list of supplies to pull from the inventory based on the plurality of potential surgical supplies; and
cause activation of a particular location indicator for a particular supply included in the list of supplies based on the particular supply being included in the list of supplies to pull, the particular location indicator indicating a location of the particular supply in a storage area of the plurality of surgical supplies.

25. The surgical supply inventory management system of claim 24, wherein the particular location indicator includes a light.

26. The surgical supply inventory management system of claim 25, wherein the light is a particular color that corresponds to a particular user.

27. The surgical supply inventory management system of claim 25, wherein the light is a particular color that corresponds to a particular surgery.

28. The surgical supply inventory management system of claim 17, wherein the surgery data includes one or more of:
surgeon information;
procedure information;
facility information; or
patient information.

29. The surgical supply inventory management system of claim 17, wherein the inventory data includes one or more of:
current stock levels of surgical supply items included in the inventory;
expiration dates of surgical supply items included in the inventory;
unique identifiers for individual surgical supply items; or
product information for individual surgical supply items.

30. The surgical supply inventory management system of claim 17, wherein the inventory control system is further configured to:
assign a particular item of the plurality of surgical supplies to a particular surgery; and
transition the particular item from an “available” status designation to an “allocated” status designation in the inventory in response to the particular item being assigned to the particular surgery.

31. The surgical supply inventory management system of claim 30, wherein the inventory control system is further configured to transition the particular item from the “allocated” status designation to a “used” status designation in the inventory in response to the particular item being used in the particular surgery.
